(19) United States
(12) Patent Application Publication
(10) Pub. No.: US 2024/0065337 A1
(43) Pub. Date: Feb. 29, 2024

(54) AEROSOL DELIVERY DEVICE WITH ACTUATABLE IGNITOR CONTACTS AND DUAL-PURPOSE SLIDER ACTUATOR

(71) Applicant: R.J. Reynolds Tobacco Company, Winston-Salem, NC (US)

(72) Inventors: Jared Aller, Winston-Salem, NC (US); Thaddeus Jackson, Summerfield, NC (US); Karen H. Cleckley, Kernersville, NC (US); Daniel W. Rennecker, Naperville, IL (US); Matthew J. Nettenstrom, Barlett, IL (US); Steven M. Schennum, Plainfield, IL (US)

(21) Appl. No.: 17/898,911

(22) Filed: Aug. 30, 2022

(51) Int. Cl. A24F 40/60; A24D 1/22; A24F 7/02; A24F 40/42; A24F 40/90

(52) U.S. Cl. CPC A24F 40/60; A24D 1/22; A24F 7/02; A24F 40/42; A24F 40/90

(57) ABSTRACT

The present disclosure is directed to an aerosol delivery device for use with a removable and replaceable cartridge and including actuatable ignitor contacts. The device includes a holder defining a receiving chamber configured to receive the cartridge, the cartridge comprising an ignitable heat source and a substrate portion comprising an aerosol precursor composition. The device further includes a first aerosol passageway, a mouthpiece defining a second aerosol passageway, and a power source disposed therein. The actuatable ignitor contacts are configured to engage with the ignitable heat source when the removable cartridge is secured within the receiving chamber and are movable between a first position spaced apart from the ignitable heat source and a second position contacting the ignitable heat source. The device may also include a dual-purpose actuator assembly coupled to the holder and configured to ignite the ignitable heat source and eject the removable cartridge.

### FIELD OF THE DISCLOSURE

The present disclosure relates to aerosol delivery devices and systems, such as smoking articles; and more particularly, to aerosol delivery devices and systems that utilize heat sources, such as combustible carbon-based ignition sources, for the production of an aerosol (e.g., smoking articles for purposes of yielding components of tobacco, tobacco extracts, nicotine, synthetic nicotine, non-nicotine flavoring, and other materials in an inhalable form, commonly referred to as heat-not-burn systems or electronic cigarettes). Components of such articles may be made or derived from tobacco, or those articles may be characterized as otherwise incorporating tobacco for human consumption, and which may be capable of vaporizing components of tobacco and/or other tobacco related materials to form an inhalable aerosol for human consumption.

### BACKGROUND

Many smoking articles have been proposed through the years as improvements upon, or alternatives to, smoking products based upon combusting tobacco. Example alternatives have included devices wherein a solid or liquid fuel is combusted to transfer heat to tobacco or wherein a chemical reaction is used to provide such heat source. Examples include the smoking articles described in U.S. Pat. No. 9,078,473 to Worm et al., which is incorporated herein by reference in its entirety.

The point of the improvements or alternatives to smoking articles typically has been to provide the sensations associated with cigarette, cigar, or pipe smoking, without delivering considerable quantities of incomplete combustion and pyrolysis products. To this end, there have been proposed numerous smoking products, flavor generators, and medicinal inhalers which utilize electrical energy to vaporize or heat a volatile material, or attempt to provide the sensations of cigarette, cigar, or pipe smoking without burning tobacco to a significant degree. See, for example, the various alternative smoking articles, aerosol delivery devices and heat generating sources set forth in the background art described in U.S. Pat. No. 7,726,320 to Robinson et al.; and U.S. Pat. App. Pub. Nos. 2013/0255702 to Griffith, Jr. et al.; and 2014/0096781 to Sears et al., which are incorporated herein by reference. See also, for example, the various types of smoking articles, aerosol delivery devices and electrically powered heat generating sources referenced by brand name and commercial source in U.S. Pat. App. Pub. No. 2015/0220232 to Bless et al., which is incorporated herein by reference. Additional types of smoking articles, aerosol delivery devices and electrically powered heat generating sources referenced by brand name and commercial source are listed in U.S. Pat. App. Pub. No. 2015/0245659 to DePiano et al., which is also incorporated herein by reference in its entirety. Other representative cigarettes or smoking articles that have been described and, in some instances, been made commercially available include those described in U.S. Pat. No. 4,735,217 to Gerth et al.; U.S. Pat. Nos. 4,922,901, 4,947,874, and 4,947,875 to Brooks et al.; U.S. Pat. No. 5,060,671 to Counts et al.; U.S. Pat. No. 5,249,586 to Morgan et al.; U.S. Pat. No. 5,388,594 to Counts et al.; U.S. Pat. No. 5,666,977 to Higgins et al.; U.S. Pat. No. 6,053,176 to Adams et al.; U.S. Pat. No. 6,164,287 to White; U.S. Pat. No. 6,196,218 to Voges; U.S. Pat. No. 6,810,883 to Felter et al.; U.S. Pat. No. 6,854,461 to Nichols; U.S. Pat. No. 7,832,476 to Hon; U.S. Pat. No. 7,513,253 to Kobayashi; U.S. Pat. No. 7,726,320 to Robinson et al.; U.S. Pat. No. 7,896,006 to Hamano; U.S. Pat. No. 6,772,756 to Shayan; U.S. Pat. App. Pub. No. 2009/0095311 to Hon; U.S. Pat. App. Pub. Nos. 2006/0196518, 2009/0126745, and 2009/0188490 to Hon; U.S. Pat. App. Pub. No. 2009/0272379 to Thorens et al.; U.S. Pat. App. Pub. Nos. 2009/0260641 and 2009/0260642 to Monsees et al.; U.S. Pat. App. Pub. Nos. 2008/0149118 and 2010/0024834 to Oglesby et al.; U.S. Pat. App. Pub. No. 2010/0307518 to Wang; and WO 2010/091593 to Hon, which are incorporated herein by reference.

Various manners and methods for assembling smoking articles that possess a plurality of sequentially arranged segmented components have been proposed. See, for example, the various types of assembly techniques and methodologies set forth in U.S. Pat. No. 5,469,871 to Barnes et al. and U.S. Pat. No. 7,647,932 to Crooks et al.; and U.S. Pat. App. Pub. Nos. 2010/0186757 to Crooks et al.; 2012/0042885 to Stone et al., and 2012/00673620 to Conner et al.; each of which is incorporated by reference herein in its entirety.

Certain types of cigarettes that employ carbonaceous fuel elements have been commercially marketed under the brand names “Premier,” “Eclipse” and “Revo” by R. J. Reynolds Tobacco Company. See, for example, those types of cigarettes described in Chemical and Biological Studies on New Cigarette Prototypes that Heat Instead of Burn Tobacco, R. J. Reynolds Tobacco Company Monograph (1988) and Inhalation Toxicology, 12:5, p. 1-58 (2000). Additionally, a similar type of cigarette has been marketed in Japan by Japan Tobacco Inc. under the brand name “Steam Hot One.”

In some instances, some smoking articles, particularly those that employ a traditional paper wrapping material, are also prone to scorching of the paper wrapping material overlying an ignitable fuel source, due to the high temperature attained by the fuel source in proximity to the paper wrapping material. This can reduce enjoyment of the smoking experience for some consumers and can mask or undesirably alter the flavors delivered to the consumer by the aerosol delivery components of the smoking articles. In further instances, traditional types of smoking articles can produce relatively significant levels of gasses, such as carbon monoxide and/or carbon dioxide, during use (e.g., as products of carbon combustion). In still further instances, traditional types of smoking articles may suffer from poor performance with respect to aerosolizing the aerosol forming component(s).

As such, it would be desirable to provide smoking articles that address one or more of the technical problems sometimes associated with traditional types of smoking articles. In particular, it would be desirable to provide a smoking article that is easy to use and that provides reusable and/or replaceable components.

### BRIEF SUMMARY

In various implementations, the present disclosure relates to aerosol delivery devices and dual-purpose slider actuators for use with removable and replaceable cartridges. The present disclosure includes, without limitation, the following example implementations.

Embodiment 1: An aerosol delivery device comprising a holder having a main body defining a proximal end and a distal end, where the main body further defines a receiving chamber configured to receive a removable cartridge comprising an ignitable heat source and a first aerosol passageway that extends through at least a portion of the main body; a mouthpiece including a first end and a longitudinally opposed second end with a second aerosol passageway extending longitudinally therebetween, wherein the first end is configured to engage with a user's mouth and the second end is configured to engage the proximal end of the holder; a power source disposed within the main body; and a dual-purpose actuator assembly coupled to the holder and configured to ignite the ignitable heat source and eject the removable cartridge.

Embodiment 2: The aerosol delivery device of the preceding Embodiment, wherein the dual-purpose actuator assembly comprises a slider body slidably disposed within the main body of the holder and defining a receptacle configured to at least partially receive the removable cartridge, the slider body configured to slide along a length of the main body in a first direction and a second direction; a spring-loaded push button coupled to the slider body so as to move the dual-purpose actuator assembly between a loading position, a lighting position, and an ejecting position, the push button configured to engage an electrical contact when in the lighting position; and a spring assembly configured to engage the slider body and bias the dual-purpose actuator assembly into the loading position, which may also be referred to as a neutral position.

Embodiment 3: The aerosol delivery device of any of Embodiments 1 and 2, or any combination thereof, wherein the dual-purpose actuator assembly comprises a slider body slidably disposed within the main body of the holder and comprising an upper track, a lower track, and a collar coupling the upper and lower tracks and defining a receptacle configured to at least partially receive the removable cartridge, the slider body configured to slide along a length of the main body in a first direction and a second direction; a spring-loaded push button engageable with the upper track of the slider body so as to move the dual-purpose actuator assembly between a loading position, a lighting position, and an ejecting position, the push button configured to engage an electrical contact when in the lighting position; and a spring assembly configured to engage the slider body and bias the dual-purpose actuator assembly into the loading position, which may also be referred to as a neutral position.

Embodiment 4: The aerosol delivery device of any of Embodiments 1 to 3, or any combination thereof, wherein the receiving chamber comprises an end cap engaged with the distal end of the main body and defining an opening configured to receive the removable cartridge; an outlet guide coupled to the slider body and slidably disposed within the main body and through the end cap, the outlet guide defining a passageway therethrough configured to pass the removable cartridge therethrough; and an inner slider seal body disposed within the receptacle of the slider body and sealingly coupled thereto, the inner slider seal body defining a cavity configured to sealingly engage and removably secure the removable cartridge therein.

Embodiment 5: The aerosol delivery device of any of Embodiments 1 to 4, or any combination thereof, further comprising a pair of actuatable ignitor contacts coupled to the dual-purpose actuator assembly and disposed proximate a distal end of the receiving chamber and configured to be engaged with the ignitable heat source when the removable cartridge is secured within the receiving chamber.

Embodiment 6: The aerosol delivery device of any of Embodiments 1 to 5, or any combination thereof, wherein the dual-purpose actuator assembly further comprises a first contact arm pivotably coupled to the main body and configured to receive one of the pair of actuatable ignitor contacts, the first contact arm actuated via contact with the lower track of the slider body when the dual-purpose actuator assembly is moved into the lighting position; and a second contact arm pivotably coupled to the main body and configured to receive the other one of the pair of actuatable ignitor contacts, the second contact arm actuated via contact with the lower track of the slider body when the dual-purpose actuator assembly is moved into the lighting position, where actuation of the first and second contact arms moves the ignitor contacts into contact with the ignitable heat source.

Embodiment 7: The aerosol delivery device of any of Embodiments 1 to 6, or any combination thereof, wherein the holder defines a slot through a surface thereof and the upper track of the slider body is sealingly engaged with the holder about the slot so that the spring-loaded push button is slidably received within the slot.

Embodiment 8: The aerosol delivery device of any of Embodiments 1 to 7, or any combination thereof, further comprising an inner housing disposed within the main body of the holder and a printed circuit board in electrical communication with the power source and comprising the electrical contact, where the inner housing is configured to receive the power source and the printed circuit board.

Embodiment 9: The aerosol delivery device of any of Embodiments 1 to 8, or any combination thereof, wherein the printed circuit board further comprises a charging port and is oriented within the inner housing so that the charging port is disposed at the proximal end of the holder.

Embodiment 10: The aerosol delivery device of any of Embodiments 1 to 9, or any combination thereof, wherein the receptacle of the slider body comprises an inner stem engageable with one end of the removable cartridge and defines a passageway therethrough for passing an aerosol generated from the removable cartridge.

Embodiment 11: The aerosol delivery device of any of Embodiments 1 to 10, or any combination thereof, wherein the outlet guide comprises a pair of opposing slots configured to allow the ignitor contacts to pass therethrough and engage the ignitable heat source.

Embodiment 12: The aerosol delivery device of any of Embodiments 1 to 11, or any combination thereof, wherein the mouthpiece is removable.

Embodiment 13: The aerosol delivery device of any of Embodiments 1 to 12, or any combination thereof, wherein the end cap is translucent and configured to allow light from the ignitable heat source to pass therethrough when lit.

Embodiment 14: The aerosol delivery device of any of Embodiments 1 to 13, or any combination thereof, wherein the device defines a complex vapor path that extends from a proximal end of the removable cartridge, through an internal passageway of the dual-purpose actuator assembly, into an inner housing and along at least channel defined by the inner housing and an inner surface of the holder, and into the mouthpiece.

Embodiment 15: The aerosol delivery device of any of Embodiments 1 to 14, or any combination thereof, wherein the inner stem is configured to engage with and eject the removable cartridge when the dual-purpose actuator assembly is moved to the ejecting position.

Embodiment 16: An aerosol delivery device comprising a holder having an upper body portion and a lower body portion, each defining a proximal end and a distal end, where the lower body portion further defines a receiving chamber disposed therein and configured to receive a removable cartridge comprising an ignitable heat source, and the upper body portion comprises a power source disposed therein, a sliding actuator assembly slidably disposed within the upper body portion and configured to eject the removable cartridge from the lower body portion and defining a first aerosol passageway, wherein the upper body portion and the lower body portion are movably coupled together (e.g., rotatably, slidably, or hingedly). The device further comprising a mouthpiece including a first end and a longitudinally opposed second end with a second aerosol passageway extending longitudinally therebetween, where the first end is configured to engage with a user's mouth and the second end is configured to engage the proximal end of the upper body portion. The mouthpiece may be removably coupled to the upper body portion. The upper body portion may also include a printed circuit board and associated electronics (e.g., a controller).

Embodiment 17: The aerosol delivery device of the preceding Embodiment further comprising a pair of actuatable ignitor contacts coupled to the lower body portion and disposed proximate the distal end thereof, where the actuatable ignitor contacts are configured to be engaged with the ignitable heat source when the removable cartridge is secured within the receiving chamber.

Embodiment 18: The aerosol delivery device of any of Embodiments 16 and 17, or any combination thereof, wherein the sliding actuator assembly comprises a tubular body defining a portion of the second aerosol passageway and configured to slide along a portion of the upper body portion in a first direction and a second direction, a first protrusion extending from an outer surface of the tubular body and configured to extend through an opening in a wall of the upper body portion to provide for moving the tubular body in the first and second directions, and a second protrusion extending from a distal end of the tubular body and configured to engage the removable cartridge so as to advance the removable cartridge through the distal end of the lower body portion when the tubular body is moved in the first or second direction. The tubular body may comprise a generally hollow body and have a cross-sectional shape, such as, for example, circular, ovoid, rectangular, triangular, or any combination of arcuate and linear elements.

Embodiment 19: The aerosol delivery device of any of Embodiments 16 to 18, or any combination thereof, wherein the sliding actuator further comprises a sealing arrangement for sealingly engaging an internal surface of the upper body portion to, for example, prevent aerosol leakage or air ingress. The sealing arrangement may include one or more O-rings disposed along and/or about the actuator.

Embodiment 20: The aerosol delivery device of any of Embodiments 16 to 19, or any combination thereof, further comprising a locking mechanism disposed on at least one of the lower body portion or the upper body portion and configured to maintain the device in a closed orientation. The locking mechanism may include one or more of magnets, friction, a snap fit, etc.

Embodiment 21: The aerosol delivery device of any of Embodiments 16 to 20, or any combination thereof, wherein the lower body portion comprises a window disposed therein and configured to provide a view of at least a portion of the removable cartridge.

Embodiment 22: The aerosol delivery device of any of Embodiments 16 to 21, or any combination thereof, wherein the lower body portion is rotatable relative to the upper body portion to expose the receiving chamber (i.e., an open configuration) and load the removable cartridge therein.

Embodiment 23: The aerosol delivery device of any Embodiment of 16 to 22, or any combination thereof, wherein one or more portions of the first protrusion, the second protrusion, or both extend into the portion of the second aerosol passageway defined the tubular body so that the aerosol generated by the cartridge is split by the one or more portions when traveling through the tubular body.

Embodiment 24: The aerosol delivery device of any Embodiment of 16 to 23, or any combination thereof, wherein the tubular body comprises a longitudinally oriented baffle extending inwardly from an interior wall thereof, where the baffle divides the aerosol traveling through tubular body.

Embodiment 25: An aerosol delivery device comprising a holder having a main body defining a proximal end and a distal end, where the main body further defines a receiving chamber configured to receive a removable cartridge comprising an ignitable heat source and a passageway that extends through at least a portion of the main body; a mouthpiece configured to engage with a user's mouth; a power source disposed within the main body; and a pair of actuatable ignitor contacts disposed proximate the distal end of the main body and configured to be engaged with the ignitable heat source when the removable cartridge is secured within the receiving chamber, wherein the ignitor contacts are movably coupled to the holder and movable between a first position spaced apart from the ignitable heat source (also referred to as an open position or configuration) and a second position contacting the ignitable heat source (also referred to as a closed position or configuration). In some implementations, the passageway is configured as a first aerosol passageway extending from proximate the receiving chamber to proximate the proximal end of the holder. Additionally, the mouthpiece may include a first end and a longitudinally opposed second end with a second aerosol passageway extending longitudinally therebetween, wherein the first end is configured to engage with the user's mouth and the second end is configured to engage the proximal end of the holder.

Embodiment 26: The aerosol delivery device of the preceding embodiment, wherein each of the actuatable ignitor contacts comprises an elongate body and a contact portion disposed at a distal end thereof, a proximal end of the elongate body pivotably coupled to the main body and wherein the contact portions are pivotable towards each other and into the second position.

Embodiment 27: The aerosol delivery device of any of Embodiments 25 and 26, or any combination thereof, wherein the pair of actuatable ignitor contacts comprises an elongate body extending between the actuatable ignitor contacts, the elongate body coupled to the main body and shaped to at least partially surround the removable cartridge, such that the actuatable ignitor contacts are deflectable towards each other and into the second position.

Embodiment 28: The aerosol delivery device of any of Embodiments 25 to 27 or any combination thereof, wherein each of the actuatable ignitor contacts comprises an elongate body and a contact portion disposed at a distal end thereof, the elongate bodies pivotably coupled to the main body and shaped to bias the ignitor contacts into the first position.

Embodiment 29: The aerosol delivery device of any of Embodiments 25 to 28, or any combination thereof, wherein the actuatable ignitor contacts are in electrical communication with the power source.

Embodiment 30: The aerosol delivery device of any of Embodiments 25 to 29, or any combination thereof further comprising an actuator assembly coupled to the holder and configured to move the actuatable ignitor contacts between the first position and the second position.

Embodiment 31: The aerosol delivery device of any of Embodiments 25 to 30, or any combination thereof, wherein the actuator assembly is configured to couple the actuatable ignitor contacts to the holder.

Embodiment 32: The aerosol delivery device of any of Embodiments 25 to 31, or any combination thereof, wherein the actuator assembly moves the ignitor contacts into electrical communication with the power source.

Embodiment 33: The aerosol delivery device of any of Embodiments 25 to 32, or any combination thereof, wherein the actuator assembly comprises a slider body slidably disposed within the main body of the holder and defining a receptacle configured to at least partially receive the removable cartridge, the slider body configured to slide along a length of the main body in a first direction and a second direction; an actuator coupled to the slider body and configured to move the actuator assembly between a loading position, a lighting position, and an ejecting position; and a spring assembly configured to engage the slider body and bias the actuator assembly into the loading or neutral position. The actuator assembly is configured to engage the actuatable ignitor contacts so as to move the ignitor contacts from the first position to the second position.

Embodiment 34: The aerosol delivery device of any of Embodiments 25 to 33, or any combination thereof, wherein the actuator assembly further comprises a spring-loaded push button coupled to the slider body and configured to be engageable with an electrical contact when in the lighting position so as to deliver electrical current to the ignitor contacts when pressed.

Embodiment 35: The aerosol delivery device of any of Embodiments 25 to 34, or any combination thereof, wherein the actuator assembly comprises a slider body slidably disposed within the main body of the holder; an actuator coupled to the slider body and configured to move the slider body along a length of the main body in a first direction and a second direction; a first contact arm pivotably coupled to the main body or the slider body and configured to receive one of the pair of actuatable ignitor contacts, the first contact arm actuated via contact with the slider body when the slider body is moved in the first direction; a second contact arm pivotably coupled to the main body or the slider body and configured to receive the other one of the pair of actuatable ignitor contacts, the second contact arm actuated via contact with the slider body when the slider body is moved in the first direction; and a biasing mechanism configured to engage the slider body and bias the slider body in the second direction. Actuation of the first and second contact arms moves the ignitor contacts from the first position to the second position.

Embodiment 36: The aerosol delivery device of any of Embodiments 25 to 35, or any combination thereof further comprising an actuator assembly coupled to the holder and configured to move the cartridge between a loading position, a lighting position, and an ejecting position, wherein the actuator assembly comprises a slider body slidably disposed within the main body of the holder and defining a receptacle configured to at least partially receive the removable cartridge, the slider body configured to slide along a length of the main body in a first direction and a second direction; an actuator coupled to the slider body and configured to move the actuator assembly between the loading position, the lighting position, and the ejecting position; and a spring assembly configured to engage the slider body and bias the actuator assembly into the loading and/or lighting position.

Embodiment 37: The aerosol delivery device of any of Embodiments 25 to 36, or any combination thereof further comprising an actuator assembly comprising a first button assembly movably disposed within the holder proximate a first one of the pair of actuatable ignitor contacts; a first contact arm pivotably coupled to the main body and configured to receive the elongate body of the first ignitor contact, the first contact arm actuated via contact with the first button assembly so as to pivot the first ignitor contact from its first position to its second position; a second button assembly movably disposed within the holder proximate the second one of the pair of actuatable ignitor contacts, the second button assembly oriented substantially in opposition to the first button assembly; and a second contact arm pivotably coupled to the main body and configured to receive the elongate body of the second ignitor contact, the second contact arm actuated via contact with the second button assembly so as to pivot the second ignitor contact from its first position to its second position.

Embodiment 38: The aerosol delivery device of any of Embodiments 25 to 37, or any combination thereof, wherein actuation of the button assemblies delivers electrical energy to the ignitable heat source.

Embodiment 39: The aerosol delivery device of any of Embodiments 25 to 38, or any combination thereof, wherein the actuator assembly comprises a first button assembly movably disposed within the holder proximate a first one of the pair of actuatable ignitor contacts and a second button assembly movably disposed within the holder proximate the second one of the pair of actuatable ignitor contacts, the second button assembly oriented substantially in opposition to the first button assembly. Actuation of the button assemblies moves the first and second actuatable ignitor contacts from their first positions to their second positions. By virtue of their placement, the buttons may be easily actuated simultaneous.

Embodiment 40: The aerosol delivery device of any of Embodiments 25 to 39, or any combination thereof further comprising a first contact arm pivotably coupled to the main body and configured to pivotably couple the first ignitor contact to the main body and a second contact arm pivotably coupled to the main body and configured to pivotably couple the second ignitor contact to the main body; wherein the first and second button assemblies are in contact with the first and second contact arms, respectively, such that actuation of the button assemblies pivots the first and second ignitor contacts into their second positions.

Embodiment 41: The aerosol delivery device of any of Embodiments 25 to 40 or any combination thereof, wherein the actuator assembly further comprises a biasing mechanism configured to engage the first and second button assemblies and bias the button assemblies outwardly into a neutral position, i.e., so that the actuatable ignitor contacts are in their first position.

Embodiment 42: The aerosol delivery device of any of Embodiments 25 to 41, or any combination thereof, wherein each of first and second button assemblies is coupled to the holder via a biasing mechanism configured to maintain the first and second button assemblies in a neutral position.

Embodiment 43: The aerosol delivery device of any of Embodiments 25 to 43, or any combination thereof further comprising an inner housing disposed within the main body of the holder and a printed circuit board in electrical communication with the power source; wherein the inner housing is configured to receive the power source and the printed circuit board.

Embodiment 44: The aerosol delivery device of any of Embodiments 25 to 44, or any combination thereof, wherein the printed circuit board further comprises a charging port and the printed circuit board is oriented within the inner housing so that the charging port is accessible via one end of the holder.

Embodiment 45: An aerosol delivery device comprising a holder comprising an upper body portion and a lower body portion, each defining a proximal end and a distal end, where the lower body portion further defines a receiving chamber disposed in the proximal end thereof and configured to receive a removable cartridge comprising an ignitable heat source and a first aerosol passageway and the upper body portion comprises a power source disposed therein and defines a second aerosol passageway therethrough, wherein the upper body portion and the lower body portion are movably coupled together; a mouthpiece configured to engage with a user's mouth; and a pair of actuatable ignitor contacts movably coupled to the lower body portion and disposed proximate the distal end thereof, wherein the actuatable ignitor contacts are configured to be engageable with the ignitable heat source when the removable cartridge is secured within the receiving chamber and movable between a first position spaced apart from the ignitable heat source and a second position contacting the ignitable heat source. The mouthpiece may include a first end and a longitudinally opposed second end with a third aerosol passageway extending longitudinally therebetween, wherein the first end is configured to engage with the user's mouth and the second end is configured to engage the proximal end of the upper body portion.

Embodiment 46: The aerosol delivery device of preceding embodiment further comprising an actuator assembly coupled to the lower body portion and configured to move the actuatable ignitor contacts between the first position and the second position.

Embodiment 47: The aerosol delivery device of any of Embodiments 45 and 46, or any combination thereof, wherein each of the actuatable ignitor contacts comprises an elongate body and a contact portion disposed at a distal end thereof, a proximal end of the elongate body pivotably coupled to the lower body portion and wherein the contact portions are pivotable towards each other and into the second position.

Embodiment 48: The aerosol delivery device of any of Embodiments 45 to 47, or any combination thereof, wherein the pair of actuatable ignitor contacts comprises an elongate body extending between the actuatable ignitor contacts, the elongate body coupled to the main body and shaped to at least partially surround the removable cartridge, such that the actuatable ignitor contacts are deflectable towards each other and into the second position.

Embodiment 49: The aerosol delivery device of any of Embodiments 45 to 48, or any combination thereof, wherein the actuatable ignitor contacts are in electrical communication with the power source.

Embodiment 50: The aerosol delivery device of any of Embodiments 45 to 49, or any combination thereof, wherein the actuator assembly comprises a first button assembly movably disposed within the lower body portion proximate a first one of the pair of actuatable ignitor contacts and a second button assembly movably disposed within the lower body portion proximate the second one of the pair of actuatable ignitor contacts, the second button assembly oriented substantially in opposition to the first button assembly. Actuation of the button assemblies moves the first and second actuatable ignitor contacts from their first positions to their second positions.

Embodiment 51: The aerosol delivery device of any of Embodiments 45 to 50, or any combination thereof, wherein each of first and second button assemblies is coupled to the holder via a biasing mechanism configured to maintain the first and second button assemblies in a neutral position.

Embodiment 52: The aerosol delivery device of any of Embodiments 45 to 51, or any combination thereof, wherein the lower body portion comprises a window disposed therein and configured to provide a view of at least the pair of actuatable ignitor contacts.

Embodiment 53: An aerosol delivery device comprising a holder having a main body defining a proximal end and a distal end, the main body further defining a receiving chamber configured to receive a removable cartridge comprising an ignitable heat source and a first passageway extending at least partially therethrough; a removable mouthpiece assembly configured to engage the proximal end of the holder, the mouthpiece assembly defined by a first end and a longitudinally opposed second end and defining a second passageway extending therethrough, wherein the first end is configured to engage with a user's mouth and the second end is configured to sealingly engage and removably secure the removable cartridge therein, where the second end is disposable within the first passageway of the main body so as to position at least a portion of the cartridge within the receiving chamber; a power source disposed within the main body; and a pair of actuatable ignitor contacts disposed proximate the distal end of the main body and configured to be engaged with the ignitable heat source when the removable cartridge is secured within the receiving chamber, wherein the ignitor contacts are movably coupled to the holder and movable between a first position spaced apart from the ignitable heat source and a second position contacting the ignitable heat source.

Embodiment 54: The aerosol delivery device of the preceding embodiment further comprising a first biasing mechanism engaged with the ignitor contacts and configured to bias the ignitor contacts into the first position.

Embodiment 55: The aerosol delivery device of any of Embodiments 53 and 54, or any combination thereof, wherein each of the actuatable ignitor contacts comprises an elongate body and a contact portion disposed at a distal end thereof, the elongate body pivotably coupled to an end cap disposed within the distal end of the holder and wherein the contact portions are pivotable towards each other and into the second position.

Embodiment 56: The aerosol delivery device of any of Embodiments 53 to 55, or any combination thereof, further comprising an actuator assembly disposed within the holder and configured to actuate the ignitor contacts between the first position and the second position.

Embodiment 57: The aerosol delivery device of any of Embodiments 53 to 56, or any combination thereof, wherein the actuator assembly comprises a solenoid in electrical communication with the power source and a collar engageable with the solenoid and a proximal end of each of the elongate bodies of the ignitor contacts, wherein the collar is configured to be advanced into contact with the elongate bodies when the solenoid is energized so as to pivot the ignitor contacts into the second position.

Embodiment 58: The aerosol delivery device of any of Embodiments 53 to 57, or any combination thereof, wherein the collar is configured to be retracted from the elongate bodies when the solenoid is de-energized so that the ignitor contacts return to the first position.

Retraction may be accomplished by the first biasing mechanism, a spring-returned solenoid, or a dual directional solenoid (e.g., energized to provide linear motion in both directions).

Embodiment 59: The aerosol delivery device of any of Embodiments 53 to 58, or any combination thereof, further comprising a printed circuit board disposed within the holder and in electrical communication with the ignitor contacts and a switch in electrical communication with the power source and the printed circuit board and configured to electrically couple the power source to the ignitor contacts.

Embodiment 60: The aerosol delivery device of any of Embodiments 53 to 59, or any combination thereof, wherein the switch is also configured to electrically couple the power source to the solenoid. In some implementations, the solenoid may be powered via other means (e.g., pneumatic).

Embodiment 61: The aerosol delivery device of any of Embodiments 53 to 60, or any combination thereof, wherein the removable mouthpiece assembly comprises a first portion comprising an elongate body defined by a first end and a longitudinally opposed second end and defining at least a portion of the second passageway; a second portion defined by a first end and a longitudinally opposed second end and defining a first aerosol passageway therethrough, wherein the first end of the second portion is configured to engage with a user's mouth and the second end is partially disposed within the second passageway in the first portion of the mouthpiece assembly proximate the first end of the first portion; a slider body defined by a first end and a longitudinally opposed second end and defining a second aerosol passageway therethrough, wherein the slider body is slidably disposed within the first portion of the mouthpiece assembly and the first end of the slider body is coupled to the second end of the second portion of the mouthpiece assembly and the second end of the slider body is configured to engage the removable cartridge; an inner tubular body defined by a first end and a longitudinally opposed second end and defining a receptacle comprising a second biasing mechanism disposed therein, the receptacle configured to at least partially receive the slider body, the slider body slidably coupled to the inner tubular body, wherein the second end of the inner tubular body is configured to engage the first portion of the mouthpiece proximate the second end of the first portion and the second biasing mechanism is configured to maintain the slider body in a loading configuration; and an outlet guide coupled to the second end of the first portion of the mouthpiece and partially received within the second end of the inner tubular body, wherein the outlet guide defines a cavity configured to sealingly engage and removably secure the removable cartridge therein.

Embodiment 62: The aerosol delivery device of any of Embodiments 53 to 61 or any combination thereof, wherein the holder further comprises an inner housing disposed within the main body of the holder and defining one or more receptacles therein, with a proximal end of the inner housing coupled to the proximal end of the holder and defining a first opening therethrough; an outer tubular body disposed within a first one of the receptacles and defining at least a portion of the first passageway and configured to slidably receive the mouthpiece assembly therein; an end cap coupled to the distal end of the holder and defining a second opening in communication with the first passageway of the outer tubular body, the end cap configured to secure at least one of the inner housing or the outer tubular body within the holder. In some implementations, the inner housing comprises an upper inner housing and a lower inner housing.

Embodiment 63: The aerosol delivery device of any of Embodiments 53 to 62, or any combination thereof, wherein the holder comprises a retention mechanism configured to movably couple the mouthpiece assembly to the holder, such as, for example, magnetic or frictional engagement.

Embodiment 64: The aerosol delivery device of any of Embodiments 53 to 63, or any combination thereof, wherein the end cap further comprises a standoff extending into the inner housing, stand-off partially defining the receiving chamber and configured to pivotably engage the actuatable ignitor contacts.

Embodiment 65: The aerosol delivery device of any of Embodiments 53 to 64, or any combination thereof, wherein the printed circuit board further comprises a charging port and the printed circuit board is oriented within the inner housing so that the charging port is disposed through a side wall of the holder.

Embodiment 66: The aerosol delivery device of any of Embodiments 53 to 65, or any combination thereof, wherein the second end of the slider body comprises a stem configured to engage with and eject the removable cartridge upon application of a force to the second portion of the mouthpiece.

Embodiment 67: The aerosol delivery device of any of Embodiments 53 to 66, or any combination thereof, wherein the second end of the second portion of the mouthpiece is configured to engage the first end of the slider body via at least one of complementary-threaded surfaces for a screw-type engagement, a press-fit engagement, a snap-fit engagement, or a magnetic engagement.

Embodiment 68: The aerosol delivery device of any of Embodiments 53 to 67, or any combination thereof, wherein the second end of the inner tubular body is configured to engage an inner surface of the first portion of the mouthpiece via at least one of complementary-threaded surfaces for a screw-type engagement, a press-fit engagement, a snap-fit engagement, or a magnetic engagement.

Embodiment 69: The aerosol delivery device of any of Embodiments 53 to 68, or any combination thereof, wherein the outlet guide is configured to engage the second end of the first portion of the mouthpiece via at least one of complementary-threaded surfaces for a screw-type engagement, a press-fit engagement, a snap-fit engagement, or a magnetic engagement.

Embodiment 70: The aerosol delivery device of any of Embodiments 1 to 69, or any combination thereof, further comprising the removable cartridge, wherein the cartridge includes a substrate material having an aerosol precursor composition configured to form an aerosol upon application of heat thereto.

Embodiment 71: The aerosol delivery device of any of Embodiments 1 to 70, or any combination thereof, wherein the holder comprises a window disposed therein and configured to provide a view of at least a portion of the removable cartridge.

Embodiment 72: The aerosol delivery device of any of Embodiments 1 to 71, or any combination thereof further comprising an indicator configured to indicate a state of the device, such as, for example, a light emitting diode to indicate on/off, charging, low battery, etc.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described the disclosure in the foregoing general terms, reference will now be made to the accompanying drawings, which are not necessarily drawn to scale, and wherein:

FIGS. 1A and 1B illustrate a perspective view and a cross-sectional perspective view, respectively, of an aerosol delivery device comprising a pair of actuatable ignitor contacts and a dual-purpose slider actuator, according to one implementation of the present disclosure;

FIG. 2 illustrates an exploded view of the aerosol delivery device of FIG. 1A, according to one implementation of the present disclosure;

FIG. 3A illustrates a cross-sectional side view of the aerosol delivery device of FIG. 1A, according to one implementation of the present disclosure;

FIG. 3B illustrates an enlarged portion of the cross-sectional side view of the aerosol delivery device of FIG. 3A, according to one implementation of the present disclosure;

FIGS. 4A-4C illustrate a series of cross-sectional side views of the aerosol delivery device of FIG. 1A in various states of operation, according to one implementation of the present disclosure;

FIGS. 5A-5C illustrate a series of enlarged cross-sectional views of a cartridge end of an aerosol delivery device comprising a pair of actuatable ignitor contacts and a dual-purpose slider actuator, with a removable cartridge being loaded therein, according to one implementation of the present disclosure;

FIGS. 6A and 6B illustrate enlarged cross-sectional views of a cartridge end of an aerosol delivery device comprising a pair of actuatable ignitor contacts and a dual-purpose slider actuator during ignition, according to one implementation of the present disclosure;

FIGS. 7A and 7B illustrate enlarged cross-sectional views of a cartridge end of an aerosol delivery device comprising a pair of actuatable ignitor contacts and a dual-purpose slider actuator, with a removable cartridge being ejected therefrom, according to one implementation of the present disclosure;

FIGS. 8A and 8B illustrate a perspective view and a cross-sectional perspective view, respectively, of another aerosol delivery device comprising a pair of actuatable ignitor contacts, according to one implementation of the present disclosure;

FIG. 9 illustrates an exploded perspective view of the aerosol delivery device of FIG. 8A, according to one implementation of the present disclosure;

FIG. 10A illustrates an enlarged cross-sectional view of a portion of a cartridge end of the aerosol delivery device of FIG. 8A with the actuatable ignitor contacts unactuated, according to one implementation of the present disclosure;

FIG. 10B illustrates an enlarged portion of the cross-sectional side view of the aerosol delivery device of FIG. 10A with a cartridge fully inserted therein, according to one implementation of the present disclosure;

FIG. 11A illustrates an enlarged perspective view a portion of a cartridge end of the aerosol delivery device of FIG. 8A with the actuatable ignitor contacts actuated, according to one implementation of the present disclosure;

FIG. 11B illustrates an enlarged plan view of the of the cartridge end of the aerosol delivery device of FIG. 11A, according to one implementation of the present disclosure;

FIGS. 12A and 12B illustrate a perspective view and a cross-sectional perspective view, respectively, of yet another aerosol delivery device comprising a slider actuator assembly, according to one implementation of the present disclosure;

FIG. 13 illustrates an exploded perspective view of the aerosol delivery device of FIG. 12A, according to one implementation of the present disclosure;

FIGS. 14A-14C illustrate a series of cross-sectional side views of the aerosol delivery device of FIG. 12A in various states of operation, according to one implementation of the present disclosure;

FIGS. 15A and 15B illustrate a perspective view and a cross-sectional perspective view, respectively, of still another aerosol delivery device comprising a pair of actuatable ignitor contacts, according to one implementation of the present disclosure;

FIG. 16 illustrates an exploded perspective view of the aerosol delivery device of FIG. 15A, according to one implementation of the present disclosure;

FIGS. 17A-17D illustrate a series of perspective views of the aerosol delivery device of FIG. 15A in various states of operation, according to one implementation of the present disclosure;

FIGS. 18A-18C illustrate a series of enlarged perspective end views of a cartridge end of the aerosol delivery device of FIG. 15A, according to one implementation of the present disclosure;

FIGS. 19 and 20 illustrate a front view and a side view, respectively, of an alternative aerosol delivery device comprising a removable mouthpiece and a removable cartridge, according to one implementation of the present disclosure;

FIG. 21 illustrates a partially exploded perspective view of the aerosol delivery device of FIG. 19, according to one implementation of the present disclosure;

FIG. 22 illustrates an exploded view of a mouthpiece portion of the aerosol delivery device of FIG. 19, according to one implementation of the present disclosure;

FIGS. 23A-23C illustrate a series of side views of the mouthpiece portion of the aerosol delivery device of FIG. 19 in various states of operation, according to one implementation of the present disclosure;

FIG. 24 illustrates an exploded view of a holder portion of the aerosol delivery device of FIG. 19, according to one implementation of the present disclosure;

FIG. 25A illustrates an enlarged view of the distal end of the aerosol delivery device of FIG. 19 in a neutral position, according to one implementation of the present disclosure;

FIG. 25B illustrates an enlarged end view of the aerosol delivery device of FIG. 25A, according to one implementation of the present disclosure;

FIG. 26A illustrates an enlarged view of the distal end of the aerosol delivery device of FIG. 19 in an ignition position, according to one implementation of the present disclosure;

FIG. 26B illustrates an enlarged end view of the aerosol delivery device of FIG. 26A, according to one implementation of the present disclosure;

FIGS. 27A-27C illustrate a series of perspective views of the aerosol delivery device of FIG. 19 in various states of operation, according to one implementation of the present disclosure;

FIG. 28 illustrates a perspective view of a removable cartridge, according to one implementation of the present disclosure; and

FIG. 29 illustrates a longitudinal cross-section view of a removable cartridge, according to one implementation of the present disclosure.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably. Additionally, where multiples of the same components are described, the multiples may be referred to individually (e.g., ##a, ##b, ##c, etc.) or collectively (##).

The present disclosure provides descriptions of articles (and the assembly and/or manufacture thereof) in which a material is heated (preferably without combusting the material to any significant degree) to form an aerosol and/or an inhalable substance; such articles most preferably being sufficiently compact to be considered “hand-held” devices. In some aspects, the articles are characterized as smoking articles. As used herein, the term “smoking article” is intended to mean an article and/or device that provides many of the sensations (e.g., inhalation and exhalation rituals, types of tastes or flavors, organoleptic effects, physical feel, use rituals, visual cues such as those provided by visible aerosol, and the like) of smoking a cigarette, cigar, or pipe, without any substantial degree of combustion of any component of that article and/or device. As used herein, the term “smoking article” does not necessarily mean that, in operation, the article or device produces smoke in the sense of an aerosol resulting from by-products of combustion or pyrolysis of tobacco, but rather, that the article or device yields vapors (including vapors within aerosols that are considered to be visible aerosols that might be considered to be described as smoke-like) resulting from volatilization or vaporization of certain components, elements, and/or the like of the article and/or device. In some aspects, articles or devices characterized as smoking articles incorporate tobacco and/or components derived from tobacco.

As noted, aerosol delivery devices may provide many of the sensations (e.g., inhalation and exhalation rituals, types of tastes or flavors, organoleptic effects, physical feel, use rituals, visual cues such as those provided by visible aerosol, and the like) of smoking a cigarette, cigar or pipe that is employed by lighting and burning tobacco (and hence inhaling tobacco smoke), without any substantial degree of combustion of any component thereof. For example, the user of an aerosol delivery device in accordance with some example implementations of the present disclosure can hold and use that device much like a smoker employs a traditional type of smoking article, draw on one end of that piece for inhalation of aerosol produced by that piece, take or draw puffs at selected intervals of time, and the like.

Articles or devices of the present disclosure are also characterized as being vapor-producing articles, aerosol delivery articles, or medicament delivery articles. Thus, such articles or devices are adaptable so as to provide one or more substances in an inhalable form or state. For example, inhalable substances are substantially in the form of a vapor (e.g., a substance that is in the gas phase at a temperature lower than its critical point). Alternatively, inhalable substances are in the form of an aerosol (e.g., a suspension of fine solid particles or liquid droplets in a gas). For purposes of simplicity, the term “aerosol” as used herein is meant to include vapors, gases, and aerosols of a form or type suitable for human inhalation, whether or not visible, and whether or not of a form that might be considered to be smoke-like. In some implementations, the terms “vapor” and “aerosol” may be interchangeable. Thus, for simplicity, the terms “vapor” and “aerosol” as used to describe the disclosure are understood to be interchangeable unless stated otherwise.

Examples of suitable vapor-producing articles, aerosol delivery articles, or medicament delivery articles include vapor products, heat-not-burn products, hybrid products and the like. Vapor products are commonly known as “electronic cigarettes,” “e-cigarettes” or electronic nicotine delivery systems (ENDS), although the aerosol-generating material need not include nicotine. Many vapor products are designed to heat a liquid material to generate an aerosol. Other vapor products are designed to break up an aerosol-generating material into an aerosol without heating, or with only secondary heating. Heat-not-burn products include tobacco heating products (THPs) and carbon-tipped tobacco heating products (CTHPs), and many are designed to heat a solid material to generate an aerosol without combusting the material.

Hybrid products use a combination of aerosol-generating materials, one or a plurality of which may be heated. Each of the aerosol-generating materials may be, for example, in the form of a solid, semi-solid, liquid, or gel. Some hybrid products are similar to vapor products except that the aerosol generated from a liquid or gel aerosol-generating material passes through a second material (such as tobacco) to pick up additional constituents before reaching the user. In some example implementations, the hybrid system includes a liquid or gel aerosol-generating material, and a solid aerosol-generating material. The solid aerosol-generating material may include, for example, tobacco or a non-tobacco product.

In use, smoking articles of the present disclosure are subjected to many of the physical actions of an individual in using a traditional type of smoking article (e.g., a cigarette, cigar, or pipe that is employed by lighting with a flame and used by inhaling tobacco that is subsequently burned and/or combusted). For example, the user of a smoking article of the present disclosure holds that article much like a traditional type of smoking article, draws on one end of that article for inhalation of an aerosol produced by that article, and takes puffs at selected intervals of time.

While the systems are generally described herein in terms of implementations associated with smoking articles such as so-called “tobacco heating products,” it should be understood that the mechanisms, components, features, and methods may be embodied in many different forms and associated with a variety of articles. For example, the description provided herein may be employed in conjunction with implementations of traditional smoking articles (e.g., cigarettes, cigars, pipes, etc.), heat-not-burn cigarettes, and related packaging for any of the products disclosed herein. Accordingly, it should be understood that the description of the mechanisms, components, features, and methods disclosed herein are discussed in terms of implementations relating to aerosol delivery devices by way of example only, and may be embodied and used in various other products and methods.

Aerosol delivery devices of the present disclosure generally include a number of components provided within an outer body or shell, which may be referred to as a housing. The overall design of the outer body or shell can vary, and the format or configuration of the outer body that can define the overall size and shape of the aerosol delivery device can vary. In some example implementations, an elongated body resembling the shape of a cigarette or cigar can be formed from a single, unitary housing or the elongated housing can be formed of two or more separable bodies. For example, an aerosol delivery device can comprise an elongated shell or body that can be substantially tubular in shape and, as such, resemble the shape of a conventional cigarette or cigar. In another example, an aerosol delivery device may be substantially rectangular or have a substantially rectangular cuboid shape. In one example, all of the components of the aerosol delivery device are contained within one housing. Alternatively, an aerosol delivery device can comprise two or more housings that are joined and are separable. For example, an aerosol delivery device can possess one portion comprising a housing containing one or more reusable components (e.g., an accumulator such as a rechargeable battery and/or rechargeable supercapacitor, and various electronics for controlling the operation of that article), and removably coupleable thereto, another second portion (e.g., a mouthpiece) and/or a disposable component (e.g., a disposable flavor-containing cartridge containing aerosol precursor material, flavorant, etc.). More specific formats, configurations and arrangements of components within the single housing type of unit or within a multi-piece separable housing type of unit will be evident in light of the further disclosure provided herein. Additionally, various aerosol delivery device designs and component arrangements can be appreciated upon consideration of the commercially available electronic aerosol delivery devices.

As will be discussed in more detail below, holders of aerosol delivery devices of the present disclosure may comprise some combination of a power source (e.g., an electrical power source), at least one control component (e.g., means for actuating, controlling, regulating and ceasing power, such as by controlling electrical current flow from the power source to other components of the article—e.g., a microprocessor, individually or as part of a microcontroller, a printed circuit board (PCB) that includes a microprocessor and/or microcontroller, etc.), a lighter portion configured heat a heat source and/or substrate material of a cartridge, and a receiving chamber. Such holders may be configured to accept one or more substrate cartridges that include a substrate material capable of yielding an aerosol upon application of sufficient heat. In some implementations, the holder may include a mouthpiece portion configured to allow drawing upon the holder for aerosol inhalation (e.g., a defined airflow path through the holder such that aerosol generated can be withdrawn therefrom upon draw).

In various aspects, the heat source of a cartridge may be capable of generating heat to aerosolize a substrate material of the cartridge that comprises, for example, an extruded structure and/or substrate, a substrate material associated with an aerosol precursor composition, tobacco and/or a tobacco related material, such as a material that is found naturally in tobacco that is isolated directly from the tobacco or synthetically prepared, in a solid or liquid form (e.g., beads, sheets, shreds, a wrap), or the like. As will be described in more detail below, in some implementations, an extruded structure may comprise tobacco products or a composite of tobacco with other materials such as, for example, ceramic powder. In other implementations, a tobacco extract/slurry may be loaded into porous ceramic beads. Other implementations may use non-tobacco products. In some implementations aerosol precursor composition-loaded porous beads/powders (ceramics) may be used. In other implementations, rods/cylinders made of extruded slurry of ceramic powder and aerosol precursor composition may be used.

In some implementations, the substrate material may comprise a liquid including an aerosol precursor composition and/or a gel including an aerosol precursor composition. Some examples of liquid compositions can be found in U.S. Pat. Pub. No. US 2020/0113239 to Aller et al., which is incorporated herein by reference in its entirety. As noted above, in various implementations, one or more of the substrate materials may have an aerosol precursor composition associated therewith. For example, in some implementations the aerosol precursor composition may comprise one or more different components, such as polyhydric alcohol (e.g., glycerin, propylene glycol, or a mixture thereof). Representative types of further aerosol precursor compositions are set forth in U.S. Pat. No. 4,793,365 to Sensabaugh, Jr. et al.; U.S. Pat. No. 5,101,839 to Jakob et al.; PCT WO 98/57556 to Biggs et al.; and Chemical and Biological Studies on New Cigarette Prototypes that Heat Instead of Burn Tobacco, R. J. Reynolds Tobacco Company Monograph (1988); the disclosures of which are incorporated herein by reference. In some aspects, a substrate material may produce a visible aerosol upon the application of sufficient heat thereto (and cooling with air, if necessary), and the substrate material may produce an aerosol that is “smoke-like.” In other aspects, the substrate material may produce an aerosol that is substantially non-visible but is recognized as present by other characteristics, such as flavor or texture. Thus, the nature of the produced aerosol may be variable depending upon the specific components of the aerosol delivery component. The substrate material may be chemically simple relative to the chemical nature of the smoke produced by burning tobacco.

In some implementations, the aerosol precursor composition may incorporate nicotine, which may be present in various concentrations. The source of nicotine may vary, and the nicotine incorporated in the aerosol precursor composition may derive from a single source or a combination of two or more sources. For example, in some implementations the aerosol precursor composition may include nicotine derived from tobacco. In other implementations, the aerosol precursor composition may include nicotine derived from other organic plant sources, such as, for example, non-tobacco plant sources including plants in the Solanaceae family. In other implementations, the aerosol precursor composition may include synthetic nicotine. In some implementations, nicotine incorporated in the aerosol precursor composition may be derived from non-tobacco plant sources, such as other members of the Solanaceae family. The aerosol precursor composition may additionally, or alternatively, include other active ingredients including, but not limited to, botanical ingredients (e.g., lavender, peppermint, chamomile, basil, rosemary, thyme, eucalyptus, ginger, cannabis, ginseng, maca, and tisanes), stimulants (e.g., caffeine and guarana), amino acids (e.g., taurine, theanine, phenylalanine, tyrosine, and tryptophan) and/or pharmaceutical, nutraceutical, and medicinal ingredients (e.g., vitamins, such as B6, B12, and C and cannabinoids, such as tetrahydrocannabinol (THC) and cannabidiol (CBD)). It should be noted that the aerosol precursor composition may comprise any constituents, derivatives, or combinations of any of the above.

As noted herein, the aerosol precursor composition may comprise or be derived from one or more botanicals or constituents, derivatives, or extracts thereof. As used herein, the term “botanical” includes any material derived from plants including, but not limited to, extracts, leaves, bark, fibers, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens.

A wide variety of types of flavoring agents, or materials that alter the sensory or organoleptic character or nature of the mainstream aerosol of the smoking article may be suitable to be employed. In some implementations, such flavoring agents may be provided from sources other than tobacco and may be natural or artificial in nature. For example, some flavoring agents may be applied to, or incorporated within, the substrate material and/or those regions of the smoking article where an aerosol is generated. In some implementations, such agents may be supplied directly to a heating cavity or region proximate to the heat source or are provided with the substrate material. Example flavoring agents may include, for example, vanillin, ethyl vanillin, cream, tea, coffee, fruit (e.g., apple, cherry, strawberry, peach and citrus flavors, including lime and lemon), maple, menthol, mint, peppermint, spearmint, wintergreen, nutmeg, clove, lavender, cardamom, ginger, honey, anise, sage, cinnamon, sandalwood, jasmine, cascarilla, cocoa, licorice, and flavorings and flavor packages of the type and character traditionally used for the flavoring of cigarette, cigar, and pipe tobaccos. Syrups, such as high fructose corn syrup, may also be suitable to be employed.

As used herein, the terms “flavor,” “flavorant,” “flavoring agents,” etc. refer to materials which, where local regulations permit, may be used to create a desired taste, aroma, or other somatosensorial sensation in a product for adult consumers. They may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some implementations, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor comprises flavor components extracted from cannabis.

In some implementations, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

Flavoring agents may also include acidic or basic characteristics (e.g., organic acids, such as levulinic acid, succinic acid, pyruvic acid, and benzoic acid). In some implementations, flavoring agents may be combinable with the elements of the substrate material if desired. Example plant-derived compositions that may be suitable are disclosed in U.S. Pat. No. 9,107,453 and U.S. Pat. App. Pub. No. 2012/0152265 both to Dube et al., the disclosures of which are incorporated herein by reference in their entireties. Any of the materials, such as flavorings, casings, and the like that may be useful in combination with a tobacco material to affect sensory properties thereof, including organoleptic properties, such as described herein, may be combined with the substrate material. Organic acids particularly may be able to be incorporated into the substrate material to affect the flavor, sensation, or organoleptic properties of medicaments, such as nicotine, that may be able to be combined with the substrate material. For example, organic acids, such as levulinic acid, lactic acid, pyruvic acid, and benzoic acid may be included in the substrate material with nicotine in amounts up to being equimolar (based on total organic acid content) with the nicotine. Any combination of organic acids may be suitable. For example, in some implementations, the substrate material may include approximately 0.1 to about 0.5 moles of levulinic acid per one mole of nicotine, approximately 0.1 to about 0.5 moles of pyruvic acid per one mole of nicotine, approximately 0.1 to about 0.5 moles of lactic acid per one mole of nicotine, or combinations thereof, up to a concentration wherein the total amount of organic acid present is equimolar to the total amount of nicotine present in the substrate material. Various additional examples of organic acids employed to produce a substrate material are described in U.S. Pat. App. Pub. No. 2015/0344456 to Dull et al., which is incorporated herein by reference in its entirety.

The selection of such further components may be variable based upon factors such as the sensory characteristics that are desired for the smoking article, and the present disclosure is intended to encompass any such further components that are readily apparent to those skilled in the art of tobacco and tobacco-related or tobacco-derived products. See, Gutcho, Tobacco Flavoring Substances and Methods, Noyes Data Corp. (1972) and Leffingwell et al., Tobacco Flavoring for Smoking Products (1972), the disclosures of which are incorporated herein by reference in their entireties.

In other implementations, the substrate material may include other materials having a variety of inherent characteristics or properties. For example, the substrate material may include a plasticized material or regenerated cellulose in the form of rayon. As another example, viscose (commercially available as VISIL©), which is a regenerated cellulose product incorporating silica, may be suitable. Some carbon fibers may include at least 95 percent carbon or more. Similarly, natural cellulose fibers such as cotton may be suitable, and may be infused or otherwise treated with silica, carbon, or metallic particles to enhance flame-retardant properties and minimize off-gassing, particularly of any undesirable off-gassing components that would have a negative impact on flavor (and especially minimizing the likelihood of any toxic off-gassing products). Cotton may be treatable with, for example, boric acid or various organophosphate compounds to provide desirable flame-retardant properties by dipping, spraying or other techniques known in the art. These fibers may also be treatable (coated, infused, or both by, e.g., dipping, spraying, or vapor-deposition) with organic or metallic nanoparticles to confer the desired property of flame-retardancy without undesirable off-gassing or melting-type behavior.

More specific formats, configurations and arrangements of components within the non-combustible aerosol provision systems of the present disclosure will be evident in light of the further disclosure provided hereinafter. Additionally, the selection and arrangement of various non-combustible aerosol provision system components can be appreciated upon consideration of the commercially available electronic non-combustible aerosol provision systems, such as those representative products referenced in the background art section of the present disclosure.

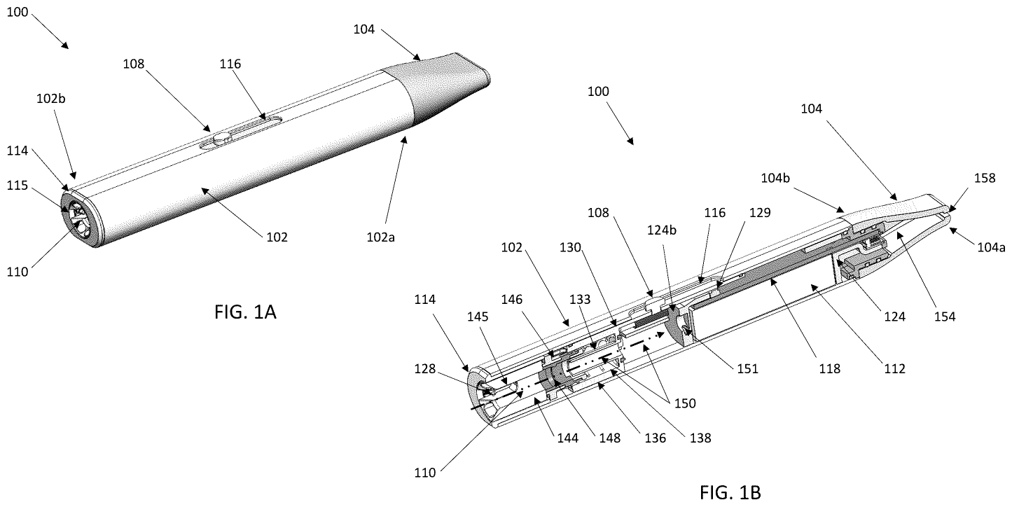

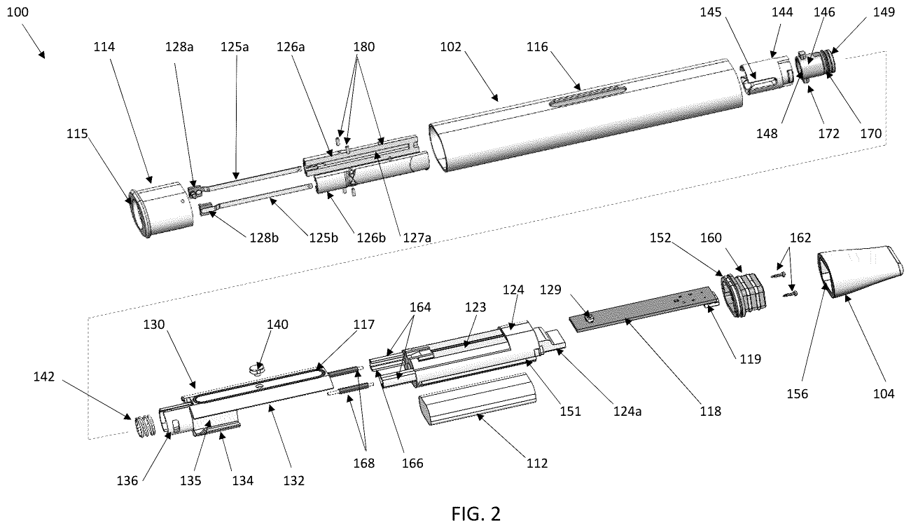

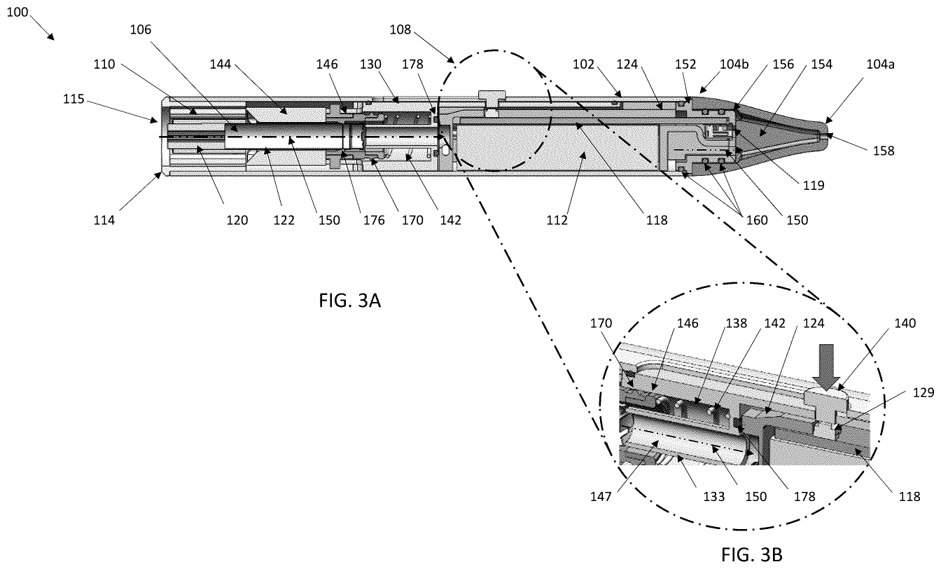

According to certain aspects of the present disclosure, it may be advantageous to provide an aerosol delivery device that is easy to use and that provides reusable and/or replaceable components. FIGS. 1A, 1, 2, 3A, and 3B illustrate one example implementation of such a device. In particular, FIGS. 1A and 1B illustrate a perspective view and a cross-sectional perspective view, respectively, of an aerosol delivery device 100 that includes a dual-purpose slider actuator assembly 108. Specifically, the aerosol delivery device 100 comprises a holder 102 having a main body defining a proximal end 102a and a distal end 102b, where the holder 102 further defines a receiving chamber 110 configured to receive a removable cartridge (106 in FIG. 3A) and an aerosol passageway 150 that extends through at least a portion of the main body. The removable cartridge 106 comprises an ignitable heat source 120 and a substrate portion 122 that includes a substrate material having an aerosol precursor composition configured to form an aerosol upon application of heat thereto. The device 100 further comprises a mouthpiece 104 having a first end 104a and a longitudinally opposed second end 104b with a second aerosol passageway 154 extending longitudinally therebetween. In the depicted implementation, the mouthpiece 104 is located proximate the proximal end 102a of the holder 102 with the first end configured to engage with a user's mouth and the second end configured to engage the proximal end of the holder 102. In the depicted implementation, the mouthpiece 104 is removable from the holder 102; however, in other implementations, the mouthpiece may be integral with the holder 102. The device 100 also includes a power source 112 disposed within the holder 102. The dual-purpose slider actuator assembly 108 is coupled to the holder 102 and configured to ignite the ignitable heat source 120 and eject the removable cartridge 106, and is described in greater detail with respect to FIGS. 2, 3A, and 3B.

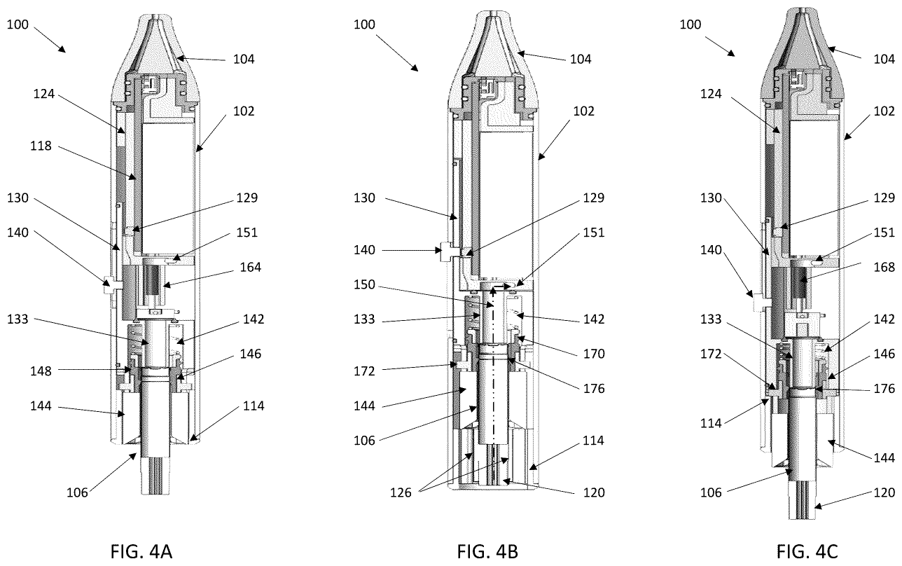

As illustrated by the exploded view of FIG. 2 and the cross-sectional side view of FIG. 3A, the dual-purpose slider actuator assembly 108 comprises a slider body 130 slidably disposed within the main body of the holder 102 and having an upper track 132, a lower track 134, and a collar 136 coupling the upper and lower tracks and defining a receptacle 138 configured to at least partially receive the removable cartridge, a spring-loaded push button 140 engaged with the upper track 132 of the slider body 130 so as to move the actuator assembly 108 between a loading position (see FIG. 4A), a lighting position (see FIG. 4B), and an ejecting position (see FIG. 4C), and a spring assembly or biasing mechanism 142 configured to engage the slider body 130 and bias the actuator assembly 108 into the loading/lighting or other “neutral” position. The slider body 130 is configured to slide along a length of the main body in a first direction and a second direction. The push button 140 is configured to engage an electrical contact 129 when in the lighting position. In one implementation, the push button 140 extends through an opening in a top surface of the upper track 132 and is slideably secured thereto. The button 140 is spring loaded so as to return to an off position after igniting the heat source. The holder 102 defines a slot 116 through a surface thereof that is sized and shaped to slidably receive the spring-loaded push button 140 therein. In some implementations, the upper track 132 of the slider body 130 is sealingly engaged with the holder about the slot 116, for example, via a gasket 117 disposed within a recess formed in the top surface of the slider body 130.

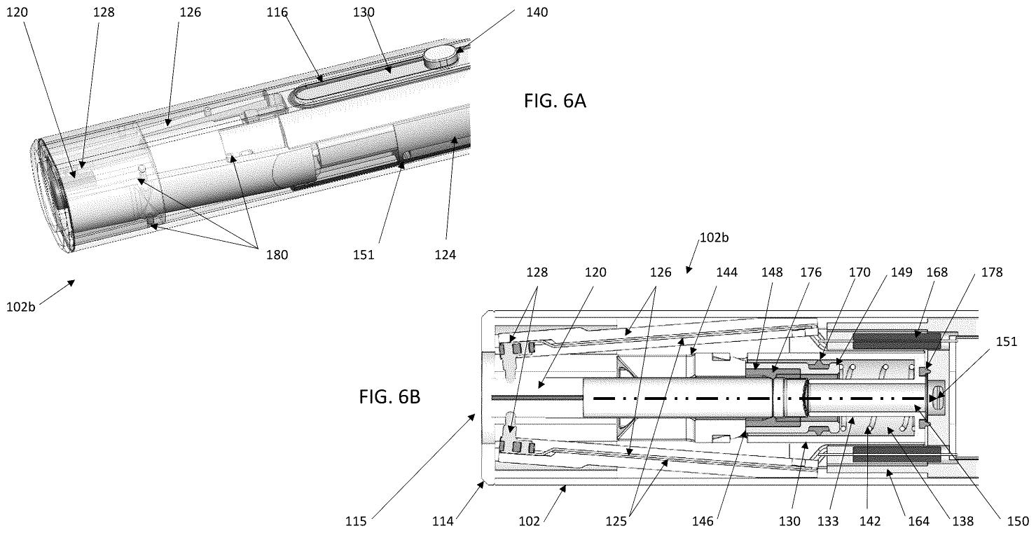

The dual-purpose slider actuator assembly 108 further comprises a pair of actuatable ignitor contacts 128A, 128b (collectively 128) disposed proximate the distal end 102a of the holder and configured to be engaged with the ignitable heat source when the removable cartridge is secured within the receiving chamber 110. The ignitor contacts 128 are coupled to the slider body 130 via a pair of contact arms 126 that are disposed within grooves 135 defined by the upper and lower tracks 132, 134. Specifically, a first contact arm 126a is pivotably coupled to the main body of the holder 102 and configured to receive one of the pair of actuatable ignitor contacts 128A in a retaining groove 127a formed within an inner surface of the contact arm 126a and a second contact arm 126b is pivotably coupled to the main body of the holder 102 and configured to receive the other one of the pair of actuatable ignitor contacts 128b in a retaining groove 127b formed within an inner surface of the second contact arm 126b. The contact arms 126 are further coupled to the sliding body 130 (e.g., via pivot pins 180) so that sliding movement of the sliding body 130 into the lighting position causes the contact arms 126 to pivot into a closed configuration where the ignitor contacts 128 move into contact with the ignitable heat source 120 (see FIG. 6B). Additionally, the contact arms 126 may be chamfered at the ends thereof that engage the slider body 130 so as to accommodate the pivoting of the arms 126 within the holder 102.

The receiving chamber 110 of the device 100 is further defined by an end cap 114 engaged with the distal end 102b of the holder and defining an opening 115 configured to receive the removable cartridge 106 therethrough, an outlet guide 144 coupled to the slider body collar 136 and slidably disposed within the main body of the holder 102 and through the end cap 114, and an inner slider seal body 146 disposed within the receptacle 138 of the slider body 130 and coupled thereto (e.g., frictionally engaged therewith via a sealing surface), the inner slider seal body 146 defining a cavity configured to sealingly engage and removably secure the removable cartridge 106 therein. For example, the inner slider seal body 146 may include an elastomeric sleeve 148 disposed therein that includes a protuberance or ring 176 extending radially inwardly from a wall of the sleeve that is configured to sealingly and frictionally engage an outer surface of the removable cartridge 106. The inner slider seal body 146 may include a leading edge or lip 149 configured to contact or otherwise engage with the spring assembly 142 with a protuberance or ring 172 disposed about an outer surface of the seal body 146 proximate an end opposite of the leading edge that is configured to engage an edge of the end cap 114 that extends within the holder 102. Additionally, the elastomeric sleeve 148 includes a protuberance or ring 170 extending radially outwardly from an exterior wall of the sleeve 148 that is configured to frictionally engage with an inner wall of the receptacle 138.

The outlet guide 144 defines a passageway that forms a portion of the receiving chamber 110 configured to pass the removable cartridge therethrough and comprises a pair of opposing slots 145 through a wall of the outlet guide 144 that are configured to allow the ignitor contacts 128 to pass therethrough to engage the ignitable heat source 120. The outlet guide 144 may be snap fit to the collar 136 via, for example, mating slots and protuberances. The collar 136 of the slider body 130 includes an inner stem 133 disposed within the receptacle 138 and engageable with one end of the removable cartridge and defining a passageway 150 therethrough for passing an aerosol generated from the removable cartridge. The end cap 114 removably engages the holder 102 via, for example, one or more of a snap-fit, interference fit, screw thread, magnetic, and/or bayonet connection. In other implementations, the end cap may be permanently engaged with the holder 102 after assembly to secure the various components therein. The end cap 114 may be translucent or transparent so as to allow light from the ignitable heat source to pass therethrough when lit. Similarly, the push button 140 may also be translucent or transparent to allow, for example, light from an LED to pass therethrough that is indicative of a state of the device 100. The operation of the dual-purpose slider actuator assembly 108 is described with respect to FIGS. 4A-4C.

The device 100 further comprises an inner housing 124 disposed within the holder 102 and defining an inner cavity 123 configured to receive the power source 112 and a printed circuit board 118 therein. The printed circuit board 118 is in electrical communication with the power source 112 and includes the electrical contact 129 disposed thereon for actuation by the slider push button 140 when the actuator assembly 108 is in the lighting position. The power source 112 is in electrical communication with the ignitor contacts 128 via a pair of wires 168 or other electrical connection mechanism. In one implementation, the wires 168 run at least partially within grooves 166 defined by a pair of fingers 164 that extend from a distal end of the inner housing 124. In addition, the distal end 124b of the inner housing sealingly engages with the slider body 130 (e.g., with a gasket or O-ring 178) when the actuator assembly is in the lighting position so as to be in fluid communication with the passageway 147 (as a portion of the first aerosol passageway 150) defined by the inner stem 133 (see FIG. 3B) and to provide the aerosol from the cartridge 106 to the aerosol passageway 150 further defined by the inner housing 124. The aerosol passageway 150 extends through the inner housing and exits via the proximal end 124a of the inner housing 124, which is in fluid communication with a second aerosol passageway 154 within the mouthpiece 104. A portion of the aerosol passageway 150 extends along and through one or more channels 151 disposed on an outer surface of the inner housing 124. The printed circuit board may further comprise a charging port 119 that is oriented on the printed circuit board so as to be disposed at the proximal end of the holder 102 and accessible by, for example, removing the mouthpiece 104.

In the depicted implementation, the mouthpiece 104 is coupled to the holder 102 via a collar 152 that sealingly engages the proximal end 102a of the holder and may be secured thereto via fasteners 162 (e.g., screws) engaged with the inner housing 124. The collar 152 includes one or more O-rings 160 disposed on an outer surface thereof and configured to sealingly engage with the mouthpiece 104, specifically the collar is received within a recess 156 formed in the distal end 104b of the mouthpiece. The collar 152 also defines a passageway therethrough that is in fluid communication with the aerosol passageway 150 of the inner housing 124 and the aerosol passageway 154 extending through the mouthpiece 104, thereby completing an aerosol path extending from the receiving chamber 110, through the sliding body 130 and the inner housing 124, and out through the opening 158 in the mouthpiece portion 104.

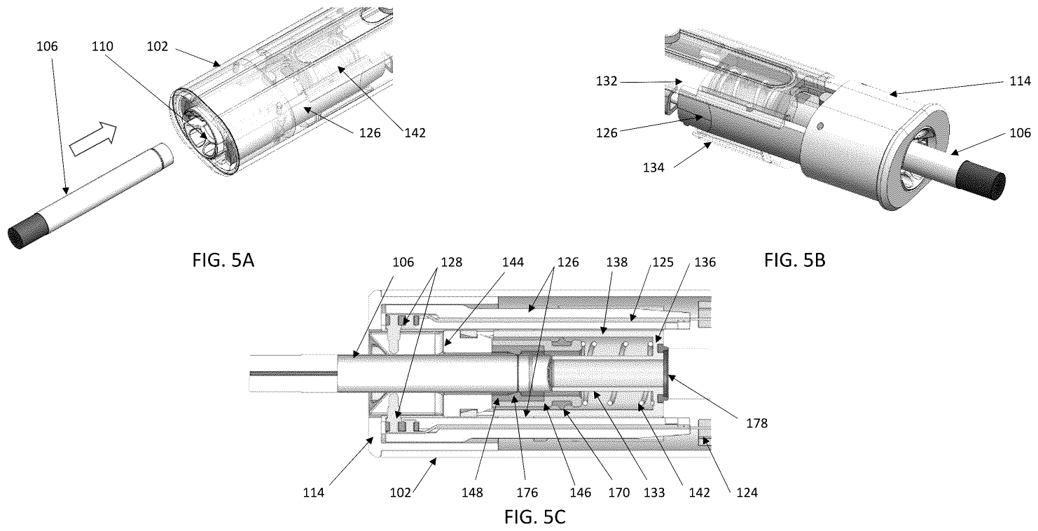

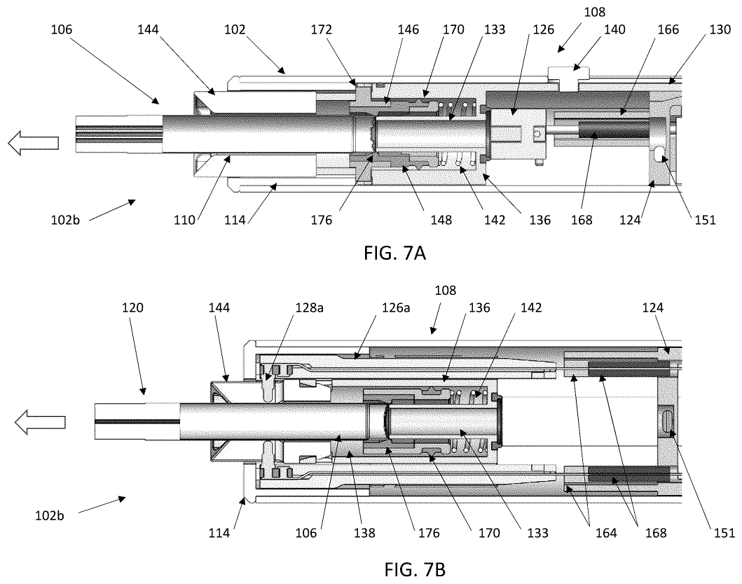

FIGS. 4A-4C illustrate a series of cross-sectional side views of the aerosol delivery device 100 in various states of operation. Specifically, FIG. 4A represents the cartridge 106 being loaded into the device 100 (i.e., the actuator assembly in the loading position) (see also FIGS. 5A-5C), FIG. 4B represents the device 100 during ignition and smoking (i.e., the actuator assembly in the ignition position) (see also FIGS. 6A and 6B), and FIG. 4C represents the cartridge 106 being ejected from the device 100 (i.e., the actuator assembly in the ejection position) (see also FIGS. 7A and 7B).

Referring to FIGS. 4A and 5A-5C, the cartridge 106 is depicted being inserted in the receiving chamber 110 of the holder 102, so as to locate the cartridge 106 into a lighting and/or use positon. In the depicted implementation, the inner slider seal body 146 forms a cartridge retention assembly configured to retain the cartridge in the receiving chamber in the lighting/use position. As previously described, the inner slider seal body 146 includes an elastomeric sleeve 148 partially disposed on an inner surface of the seal body that includes structure 176 for engaging the cartridge 106. As shown, the seal body has a generally cylindrical shape; however, the shape and size of the seal body 146 may vary to suit a particular application. In particular, the sleeve 148 comprises a silicone seal configured to frictionally engage the cartridge so that when the cartridge 106 is pushed into and fully received within the receiving chamber 110, the cartridge 106 is temporarily “locked” in place within the holder 102. In some implementations, a protuberance 176 such as a ring that extends radially inward from an interior surface of the sleeve 148 is included and is configured to frictionally and/or sealingly engage an outer surface of the removable cartridge 106 to secure the cartridge 106 within the receiving chamber 110.

In other implementations, other retaining features may be used. For example, in some implementations one or more retention spheres may form part of a cartridge retention assembly. In other implementations, a cartridge retention assembly may comprise one or more resilient members, such as, for example, one or more O-rings, and/or other retaining features that include one or more resilient features that extend into the receiving chamber in order to engage a portion of the outer surface of the cartridge. In other implementations, an outer housing of the cartridge and/or the receiving chamber may include one or more protrusions and/or spring features and corresponding detent features configured to retain the cartridge in the receiving chamber (e.g., a spring-loaded latching mechanism). In still other implementations, an inner surface of the receiving chamber may have a decreasing diameter (and/or one or more portions having a decreased diameter) that may be configured to retain the cartridge in the receiving chamber. In other implementations, the holder may include actively retractable features (e.g., features that are actively retractable by a user) configured to engage the cartridge to retain it in the receiving chamber. In other implementations, the holder may include one or more wedge features configured to engage and retain the cartridge in the receiving chamber. In still other implementations, one or more other features of the cartridge and/or one or more features of the holder may create a releasable connection between the receiving chamber and the cartridge. For example, in some implementations, the cartridge and the receiving chamber may have a releasable screw-type connection. In still other implementations, the cartridge may be retained in the receiving chamber via magnetic force. For example, in some implementations the outer housing of the cartridge may be made of a ferromagnetic material, and the receiving chamber may include one or more magnets. Combinations of two or more of these retaining features may also be used.

In various implementations, one or more components of a cartridge retention assembly may be made of any material, including for example, but not limited to, metal or plastic materials. For example, some implementations may include one or more components of a cartridge retention assembly that are made of a metal material such as, for example, stainless steel, aluminum, brass, copper, silver, gold, bronze, titanium, various alloys, etc. In some implementations, one or more components of a cartridge retention assembly may be made of a moldable plastic material such as, for example, polycarbonate, polyethylene, acrylonitrile butadiene styrene (ABS), polyamide (Nylon), or polypropylene. In some implementations, one or more components of a cartridge retention assembly may be made of a different material, such as, for example, a different plastic material, a different metal material, a graphite material, a glass material, a ceramic material, a natural material (such as, but not limited to, a wood material), a composite material, or any combinations thereof.

As shown in FIGS. 4A and 5C, the actuator assembly 108 is in a forward orientation (i.e., loading position) within the holder 102 so that the contact arms 126 are in an open configuration and the push button 140 is offset from the ignition contact 129 so that electrical current cannot be accidentally directed to the ignition contacts 128. In the open position, the contact arms 126 rest on the outlet guide 144 and are not able to pivot into contact with the ignition contacts 128 of the cartridge. With the actuator assembly 108 in the loading position, the cartridge 106 is manually inserted into the receiving chamber 110 until secured therein with a portion of the cartridge 106 (e.g., the ignitable heat source 120) extending beyond the distal end 102b of the holder 102. In some implementations, the cartridge 106 extends beyond the distal end 102b by about 5 mm to about 25 mm, preferably about 10 mm to about 20 mm, and more preferably about 14.5 mm; however, the cartridge is secured within the inner slider seal body to prevent it from falling out of the receiving chamber 110. In some implementations, the retention structure (e.g., protuberance 176) may include audible or tactile feedback to inform a user that the cartridge 106 is secured within the device 100. Additionally, the outlet guide 144 is generally positioned within the end cap 114 proximate the distal end of the holder 102 and a proximal end of the cartridge 106 is sealingly engaged with the stem 133 so that a passageway therein is in fluid communication with the aerosol passageway 150 of the inner housing 124.

Referring to FIGS. 4B, 6A, and 6B, the slider push button 140 (and actuator assembly 108) is moved towards the mouthpiece 104 to pull the cartridge 106 into the lighting or use position within the device 100. As shown in FIG. 4B, the push button 140 is aligned with the contact 129 so as to complete the electrical circuit and deliver electrical current to the ignitor contacts 128 when pressed. In the lighting/use position depicted in FIGS. 6A and 6B, the distal end of the cartridge 106 is located proximate the distal end 102b of the holder such that the entire cartridge 106 is located inside of the holder 102. In particular, in the lighting/use position of the depicted implementation, the ignitable heat source 120 portion of the cartridge 106 is also positioned proximate the distal end of the holder 102 and aligned with the ignitor contacts 128 in the lighting position. Generally, the device 100 and the cartridge are configured so that the distal end of the cartridge 106 is substantially aligned with (or, in some implementations, inserted past) the distal end of the holder 102 such that the distal end of the cartridge 106 does not extend beyond the distal end 102b of the holder. In the lighting/use position of other implementations, however, a cartridge may be received into the holder to varying degrees, and, in some implementations, the distal end of the cartridge may extend beyond (e.g., outside of) the distal end of the holder. As will be described in more detail below, in the lighting/use position the ignition contacts 128 are configured to ignite the heat source 120 of the substrate cartridge 106.

FIGS. 6A and 6B illustrate the distal end 102b of the holder and, in particular, the interface between the cartridge 106 and the sliding actuator assembly 108 and the ignitor contacts 128. As shown, the cartridge 106 is sealingly engaged with the slider body stem 133 (via inner slider seal 166) and the slider body 130 is fully retracted and sealingly engaged with the inner housing 124 (via seal 178) so as define the aerosol passageway 150 previously described. In the lighting position, the ignitable heat source 120 is ignited so that the ignited heat source aerosolizes at least a portion of the substrate portion 122 of the cartridge 106 for delivery to a user through the holder 102.

In some implementations, the opening 115 in the end cap 114 allows for the introduction of ambient air to the receiving chamber 110 and cartridge 106. However, in other implementations not depicted, the holder may include one or more apertures therein for allowing entrance of ambient air to be directed into the receiving chamber and/or the aerosol passageway (such as, for example, through the substrate cartridge and/or downstream from the substrate cartridge). Thus, when a user draws on the holder (e.g., via the mouthpiece portion thereof), air may be drawn into the receiving chamber and/or the aerosol passageway for inhalation by the user. Furthermore, when the actuator assembly 108 is moved into the lighting position, the lower track 134 of the slider body 130 forces the contact arms 126 into their closed configuration (i.e., the ignitor contacts 128 are in contact with the heat source 120). Specifically, the contact arms 126 are engaged with the slider body 130 and pivotably coupled to the holder 102 so that when the actuator assembly 108 is moved into the lighting position, the outlet guide 144 is translated toward the proximal end 102a of the holder 102. In this manner, the contact arms 126 are no longer supported in their open configuration by the outlet guide 144 and are able to pivot into the closed configuration so that the ignitor contacts 128 are in contact with the heat source 120. Once the push button 140 is actuated, the electrical circuit is completed and the electricity is delivered to the ignitor contacts 128. In some implementations, the ignitor push button 140 may be configured to activate the ignitor contacts 128 for a set time after release or the ignitor contacts will be deactivated when the ignitor push button is released.

In the depicted implementation, the location of the cartridge 106 relative to the holder 102 in the lighting position and the use position comprises a single location; however, in other implementations, there may be separate and distinct lighting and use positons. In the lighting/use position of some implementations, a cartridge may be received into the holder to varying degrees. For example, in the lighting/use position of some implementations, less than a half of the length of the cartridge may be located within the holder (e.g., less than 50%, less than 40%, less than 30%, less than 20%, less than 10%, etc.). In the lighting/use position of other implementations, approximately half of the length of the cartridge may be received into the holder. In the lighting/use position of other implementations, more than a half of the length of the cartridge may be received into the holder (e.g., more than 50%, more than 60%, more than 70%, more than 80%, more than 90%, etc.).

In some alternative implementations, the aerosol is generated by an electric heater configured to perform electric heating in which electrical energy from the power source is delivered to the heater when the actuator assembly is moved into the lighting/use position. Subjecting the aerosol-generating material (substrate 122) to heat releases one or more volatiles from the aerosol-generating material to form an aerosol. Examples of suitable forms of electric heating include resistance (Joule) heating, induction heating, dielectric and microwave heating, radiant heating, arc heating and the like. More particular examples of suitable electric heaters include resistive heating elements such as wire coils, flat plates, prongs, micro heaters or the like.

In some examples, the cartridge 106 may include a susceptor (e.g., the susceptor may be part of the substrate 122). The susceptor is a material that is heatable by penetration with a varying magnetic field generated by a magnetic field generator that may be separate from or part of the aerosol generator. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor in some examples may be both electrically-conductive and magnetic, so that the susceptor of these examples is heatable by both heating mechanisms.

In some implementations, the device 100 may include an ejection mechanism, as shown in FIGS. 4C, 7A, and 7B. Generally, the ejection mechanism is configured to eject the cartridge 106 from the distal end 102b of the holder. In one implementation, the ejection mechanism is incorporated in to the actuator assembly 108 so that when the slider push button 140 (and actuator assembly 108) is moved forward it engages or contacts one end of the cartridge 106 and pushes the cartridge out of the receiving chamber 110 and out of the aerosol delivery device 100. FIGS. 7A and 7B illustrate the distal end 102b of the holder and, in particular, the ejection of the cartridge 106 out of the receiving chamber 110 and through the opening 115 in the end cap 114. Specifically, a user can slide the actuator assembly 108 forward (e.g., using their thumb and the push button 140) so that the inner stem 133 of the slider body 130 engages the cartridge 106 pushing it out of the inner slider seal 146 so that the cartridge becomes disengaged from the cartridge retention structure 176 disposed within the inner slider seal body 146. As such, the cartridge 106 may be pulled out of or allowed to drop out of the receiving chamber 110.

As previously described, the inner slider seal body 146 and the outlet guide 144 are coupled to the slider body 130. When the slider body 130 moves forward, the outlet guide may extend beyond the end cap 114 of the device, while a portion (ring 172) of the inner slider seal body 146 engages an edge of the end cap 114 preventing the actuator assembly 108 from falling out of the holder 102. The leading edge 149 of the inner slider seal body 146 is configured to contact or otherwise engage with the spring assembly 142 so that the spring assembly 142 is compressed when the actuator assembly is moved into the ejection position, so that once the cartridge is ejected and the actuator assembly 108 is released, the actuator assembly moves to the loading position.

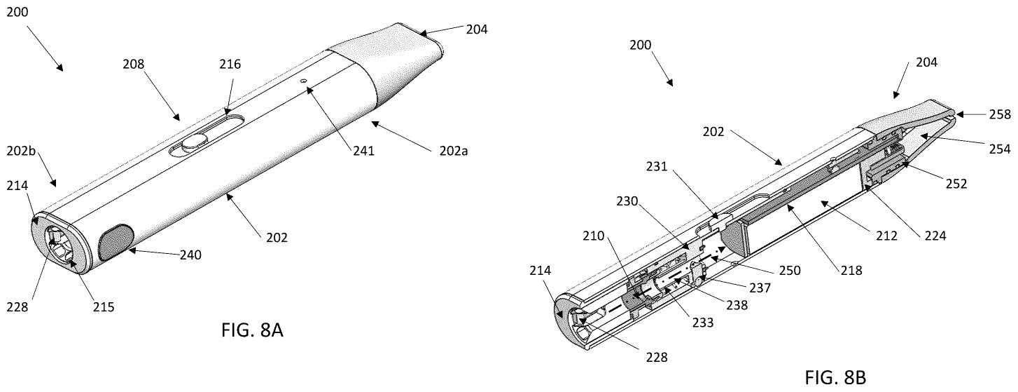

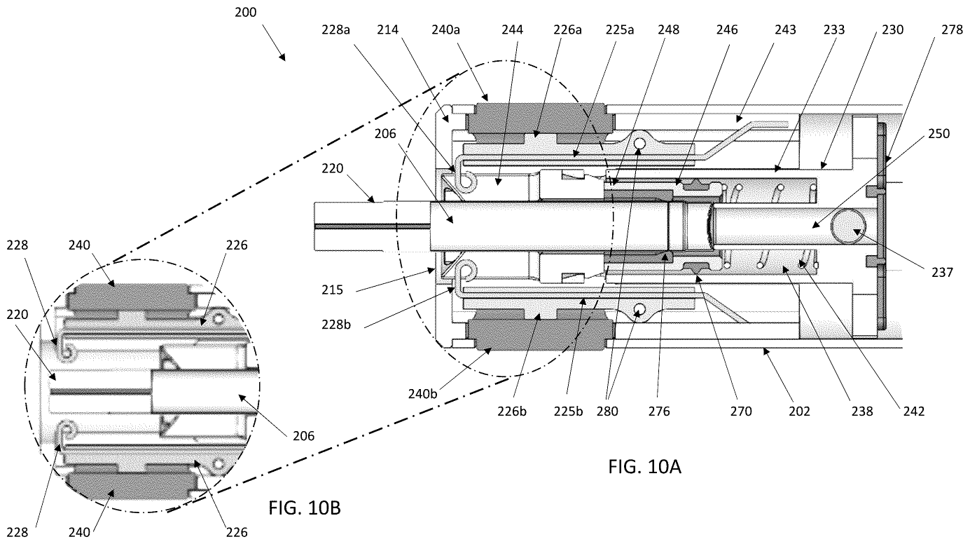

FIGS. 8A and 8B illustrate a perspective view and a cross-sectional side view, respectively, of another implementation of an aerosol delivery device 200 similar to the device 100 described with respect to FIGS. 1A and 1B. Specifically, the aerosol delivery device 200 comprises a holder 202 having a main body defining a proximal end 202a and a distal end 202b, where the holder 202 further defines a receiving chamber 210 configured to receive a removable cartridge (206 in FIG. 10A) and a passageway (e.g., a first aerosol passageway) 250 that extends through at least a portion of the main body. The removable cartridge 206 comprises an ignitable heat source 220 and a substrate portion 222 that includes a substrate material having an aerosol precursor composition configured to form an aerosol upon application of heat thereto. The device 200 also includes a pair of actuatable ignitor contacts 228 that are configured to be engageable with the ignitable heat source 220 when the removable cartridge 206 is secured within the receiving chamber 210. Specifically, the ignitor contacts 228 are configured to be movable between a first position spaced apart from the ignitable heat source 220 and a second position contacting the ignitable heat source 220.

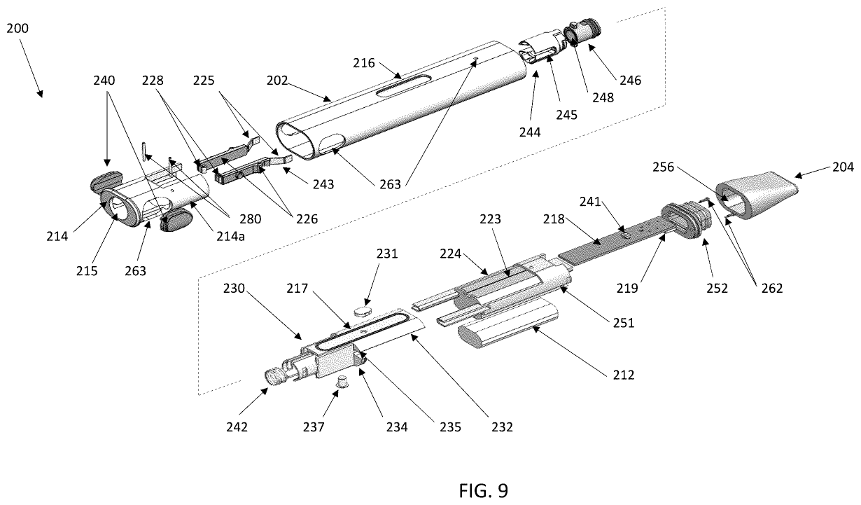

The device 200 further comprises a mouthpiece 204 having a first end 204a and a longitudinally opposed second end 204b with a second aerosol passageway 254 extending longitudinally therebetween. In the depicted implementation, the mouthpiece 204 is located proximate the proximal end 202a of the holder 202 with the first end configured to engage with a user's mouth and the second end configured to engage the proximal end of the holder 202. In the depicted implementation, the mouthpiece 204 is removable from the holder 202; however, in other implementations, the mouthpiece may be integral with the holder 202. The device 200 also includes a power source 212 disposed within the holder 202 and a slider actuator assembly 208 coupled to the holder 202 and configured to load and eject the removable cartridge 206. The actuator assembly 208 is similar to the actuator assembly 108 of FIGS. 1A and 1B and is described in greater detail with respect to FIG. 9.

As illustrated by the exploded view of FIG. 9, the slider actuator assembly 208 comprises a slider body 230 slidably disposed within the main body of the holder 202 and having an upper track 232, a lower track or protrusion 234, and a collar 236 coupling the upper and lower tracks and defining a receptacle 238 configured to at least partially receive the removable cartridge, a slider button 231 engaged with the upper track 232 of the slider body 230 so as to move the actuator assembly 208 between a loading position, a lighting position, and an ejecting position (similar to those described with respect to FIGS. 4A-4C), and a spring assembly 242 configured to engage the slider body 230 and bias the actuator assembly 208 into the loading or other “neutral” position. The slider body 230 is configured to slide along a length of the main body in a first direction and a second direction. A locating feature 237 (such as, for, example, a spring-loaded ball plunger mechanism) and one or more corresponding detents may be included to temporarily locate the actuator assembly 208 in one or more of the loading, lighting, and ejecting positions. The holder 202 defines a slot 216 through a surface thereof that is sized and shaped to slidably receive the slider button 231 therein. In some implementations, the upper track 232 of the slider body 230 is sealingly engaged with the holder about the slot 216, for example, via a gasket 217 disposed within a recess formed in the top surface of the slider body 230. The upper and lower tracks 232, 234 define a groove 235 there between for receiving elongated legs of the inner housing 224 therebetween so as to couple the inner housing 224 with the slider body.

The receiving chamber 210 of the device 200 is further defined by an end cap 214 engaged with the distal end 202b of the holder and defining an opening 215 configured to receive the removable cartridge 206 therethrough, an outlet guide 244 coupled to the slider body collar 236 and slidably disposed within the main body of the holder 202 and through the end cap 214, and an inner slider seal body 246 disposed within the receptacle 238 of the slider body 230 and coupled thereto (e.g., frictionally engaged therewith via a sealing surface), the inner slider seal body 246 defining a cavity configured to sealingly engage and removably secure the removable cartridge 206 therein. For example, the inner slider seal body 246 may include an elastomeric sleeve 248 disposed therein that includes a protuberance or ring 276 extending radially inwardly from a wall of the sleeve that is configured to frictionally and/or sealingly engage an outer surface of the removable cartridge 206. In some implementations, the elastomeric sleeve 248 may be integral with the inner slider seal body 246, such as, for example, as part of an over-molded part. The inner slider seal body 246 may include a leading edge or lip 249 configured to contact or otherwise engage with the spring assembly 242 with a protuberance or ring 272 disposed about an outer surface of the seal body 246 proximate an end opposite of the leading edge that is configured to engage an edge of the end cap 214 that extends within the holder 202. Additionally, the elastomeric sleeve 248 includes a protuberance or ring 270 extending radially outwardly from an exterior wall of the sleeve 248 that is configured to frictionally engage with an inner wall of the receptacle 238.

The outlet guide 244 defines a passageway that forms a portion of the receiving chamber 210 configured to pass the removable cartridge therethrough and comprises a pair of opposing slots 245 through a wall of the outlet guide 244 that are configured to allow the ignitor contacts 228 to pass therethrough to engage the ignitable heat source 220. The outlet guide 244 may be snap fit to the collar 236 via, for example, mating slots and protuberances. The collar 236 of the slider body 230 includes an inner stem 233 disposed within the receptacle 238 and engageable with one end of the removable cartridge and defining the passageway 250 therethrough for passing an aerosol generated from the removable cartridge. The end cap 214 removably engages the holder 202 via, for example, one or more of a snap-fit, interference fit, screw thread, magnetic, and/or bayonet connection. In other implementations, the end cap may be permanently engaged with the holder 202 after assembly to secure the various components therein. The end cap 214 may also be translucent or transparent so as to allow light from the ignitable heat source to pass therethrough when lit.

The device 200 further comprises an inner housing 224 disposed within the holder 202 and defining an inner cavity 223 configured to receive the power source 212 and a printed circuit board 218 therein. The printed circuit board 218 is in electrical communication with the power source 212 and includes the LED 241 disposed thereon for indicating when the ignitor contacts 228 are energized. The power source 212 is in electrical communication with the ignitor contacts 228 via a pair of wires or other electrical connection mechanism. In one implementation, the wires run at least partially within grooves defined by a pair of fingers that extend from a distal end of the inner housing 224. In addition, the distal end of the inner housing sealingly engages with the slider body 230 (e.g., with a gasket or O-ring 278) when the actuator assembly is in the lighting position so as to be in fluid communication with the passageway defined by the inner stem 233 and to provide the aerosol from the cartridge 206 to the first aerosol passageway 250 further defined by the inner housing 224. The first aerosol passageway 250 extends through the inner housing and exits via the proximal end of the inner housing 224, which is in fluid communication with a second aerosol passageway 254 within the mouthpiece 204. A portion of the first aerosol passageway 250 extends along and through one or more channels 251 disposed on an outer surface of or within the inner housing 224. The printed circuit board may further comprise a charging port 219 that is oriented on the printed circuit board so as to be disposed at the proximal end of the holder 202 and accessible by, for example, removing the mouthpiece 204.

In the depicted implementation, the mouthpiece 204 is coupled to the holder 202 via a collar 252 that sealingly engages the proximal end 202a of the holder and may be secured thereto via fasteners 262 (e.g., screws) engaged with the inner housing 224. The collar 252 includes one or more sealing mechanisms disposed on an outer surface thereof and configured to sealingly engage with the mouthpiece 204, specifically the collar is received within a recess 256 formed in the distal end 204b of the mouthpiece. The collar 252 also defines a passageway therethrough that is in fluid communication with the first aerosol passageway 250 of the inner housing 224 and the aerosol passageway 254 extending through the mouthpiece 204, thereby completing an aerosol path extending from the receiving chamber 210, through the sliding body 230 and the inner housing 224, and out through the opening 258 in the mouthpiece portion 204.

The pair of actuatable ignitor contacts 228a, 228b (collectively 228) are disposed proximate the distal end 202a of the holder and configured to be engageable with the ignitable heat source 220 when the removable cartridge is secured within the receiving chamber 210. The ignitor contacts 228 are coupled to a pair of contact arms 226 that are pivotably coupled to the holder 202. Each contact 228 is disposed at a distal end of an elongate body 225 and/or formed as a single piece therewith. In the depicted implementation, a relatively flat portion of each elongate body is configured to be coupled to the contact arms 226 via, for example, a snap-fit, an interference fit, or a mechanical fastener. Each contact arm 226 is secured within the holder 202 via a pivot pin 280. In some implementations, the contact arms 226 may be pinned to a body portion 214a of the end cap 214 so that the assembled parts may be inserted into the distal end of the holder 202.

Additionally, each of the proximal portions of the elongate bodies 225 comprise reverse compound bends 243 that act as leaf springs so as to maintain the ignitor contacts 228 in their first, or open, position. Specifically, the proximal ends of the elongate bodies 225 abut or are secured relative to an inner wall of the holder 202 such that the compound bend 243 provides the biasing force to the contact arms 226 to maintain the contacts 228 in the first position (see FIG. 10A). Also disposed proximate the distal end 202b of the holder 202 is a pair spring loaded push buttons 240 that are configured to actuate the ignitor contacts 228 and deliver electrical energy to the ignitable heat source 220 as described below. The push buttons 240 pass through aligned openings 263 disposed within the holder 202 and body portion 214a of the end cap 214. An additional opening 263 is disposed within a top wall of the holder 202 and aligned with the LED 241 disposed on the printed circuit board 218.

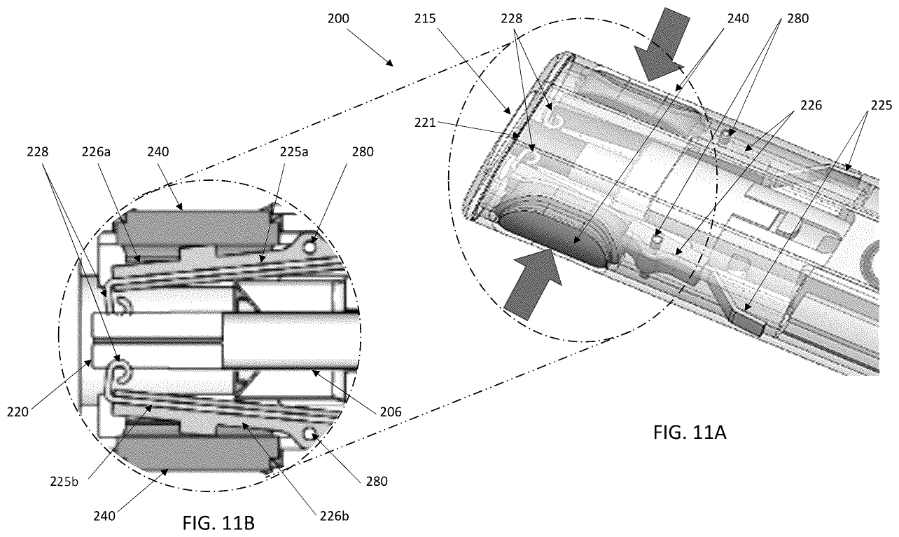

FIGS. 10A and 10B depict the device 200 in a loading or neutral position where the cartridge 206 has been introduced to the receiving chamber 210 of the device, with the cartridge 206 shown fully loaded in the detail view and the ignitor contacts 228 in a first, unactuated position where the ignitor contacts 228 are positioned near, but not in contact with, the ignitable heat source 220. FIGS. 11A and 11B depict the device 200 after the slider actuator assembly 208 has been moved to the lighting position, with the ignitor contacts 228 shown in a second, actuated position where the contacts 228 are in contact with the ignitable heat source 220. Generally, the ignitor contacts 228 are actuated between their first and second positions in a similar manner as the ignitor contacts 328 described hereinabove insofar as the heat source 220 is ignited via the aligned push buttons 240 disposed on the opposite sides of the aerosol delivery device 200. The push buttons 240 are movably coupled to the housing 202, with or without a sealing arrangement, so that a user may press both buttons simultaneously (e.g., via a pinching action) so as to pivot the contact arms 226 and by extension move the ignitor contacts 228 into contact with the ignitable heat source 220. Once released, the buttons 240 return to their original position, at least in part via a spring action from the reverse compound bends 243 of the elongate bodies 225.

Specifically, the device 200 includes the two spring loaded buttons 240 disposed on opposing sides of the holder 202 and oriented proximate the contact arms 226. In some implementations, the buttons 240 and contact arms 226 may include mating structure to provide a more positive movable engagement therebetween. As shown in FIG. 11B, the spring loaded push buttons 240 are simultaneously pressed to activate ignition. Specifically, when the buttons 240 are pressed, power is activated and the ignitor contacts 228 are pushed onto the ignitable heat source 220. Releasing the buttons 240 terminates power and the ignitor contacts 228 pull away from tip automatically (e.g., return to their first position). In some embodiments, a portion of the holder 202 may be transparent or translucent or incorporate a window that allows a user to see the glow of the ignitable heating source 220.

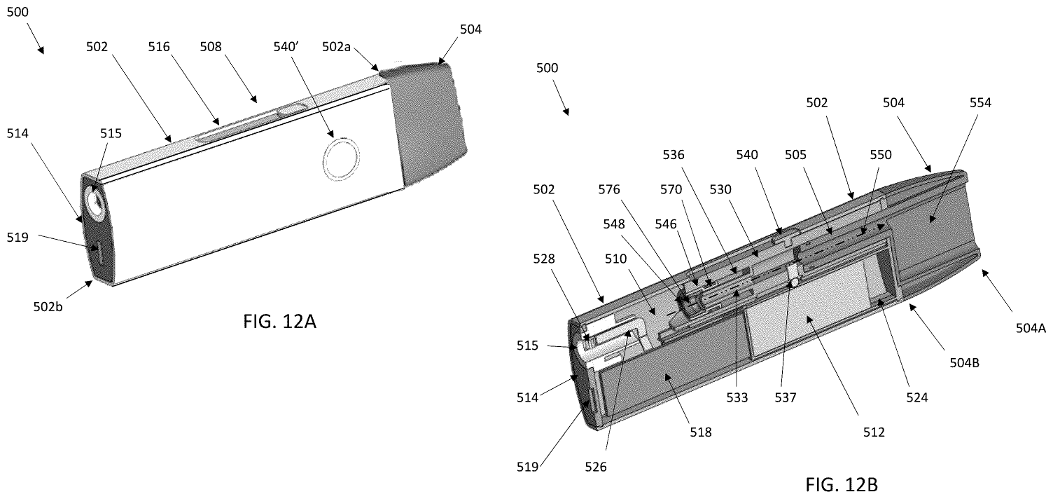

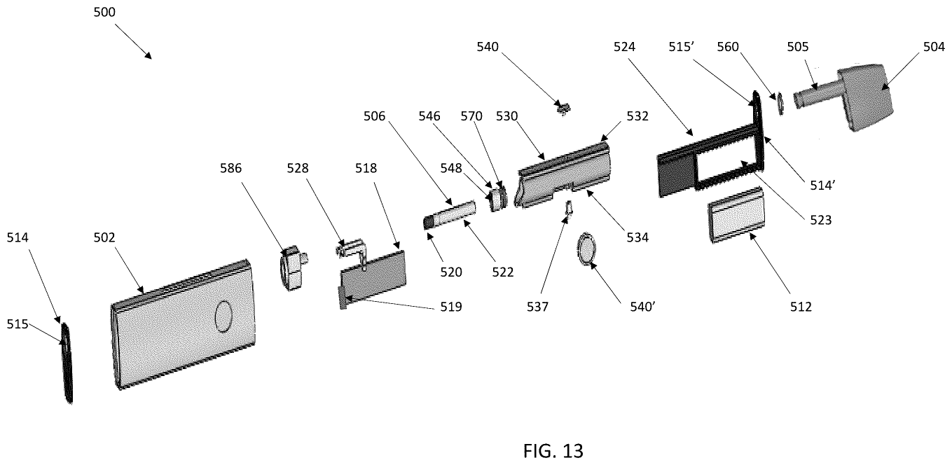

FIGS. 12A, 12B, and 13 illustrate another example implementation of an aerosol delivery device 500. In particular, FIGS. 12A and 12B illustrate a perspective view and a cross-sectional perspective view, respectively, of an aerosol delivery device 500 that includes a slider actuator assembly 508. Specifically, the aerosol delivery device 500 comprises a holder 502 having a main body defining a proximal end 502a and a distal end 502b, where the holder 502 further defines a receiving chamber 510 configured to receive a removable cartridge (506 in FIG. 13) and an aerosol passageway 550 that extends through at least a portion of the main body. The removable cartridge 506 comprises an ignitable heat source 520 and a substrate portion 522 that includes a substrate material having an aerosol precursor composition configured to form an aerosol upon application of heat thereto. The device 500 further comprises a mouthpiece 504 having a first end 504a and a longitudinally opposed second end 504b with a second aerosol passageway 554 extending longitudinally therebetween. In the depicted implementation, the mouthpiece 504 is located proximate the proximal end 502a of the holder 502 with the first end configured to engage with a user's mouth and the second end configured to engage the proximal end of the holder 502. The mouthpiece 504 includes an extension or hollow stem 505 that extends from the second end 504b of the mouthpiece and is configured to sealingly engage with the slider body 530 and partially define the aerosol passageway 550. In the depicted implementation, the mouthpiece 504 is removable from the holder 502 for, for example, cleaning or customization of the device 500 (e.g., the use of interchangeable mouthpieces). However, in other implementations, the mouthpiece 504 may be integral with the holder 502. The device 500 also includes a power source 512 disposed within the holder 502. The slider actuator assembly 508 is coupled to the holder 502 and configured to eject the removable cartridge 506, as described in greater detail below.

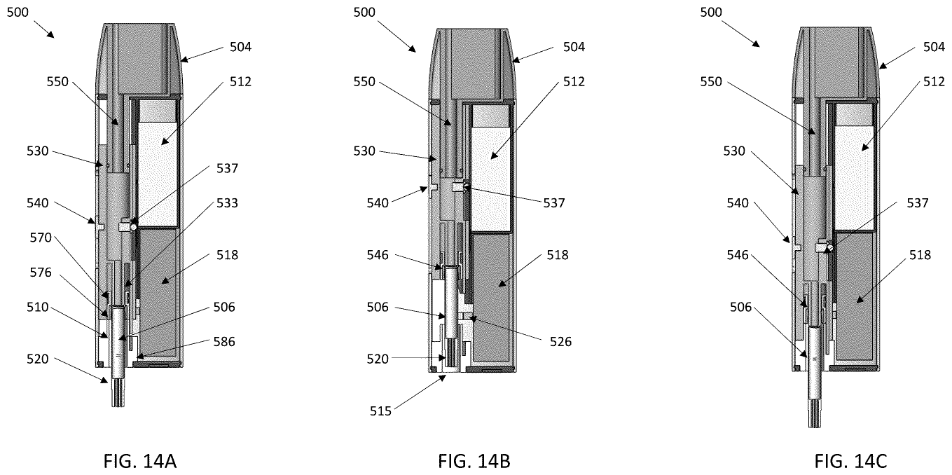

As further illustrated by the exploded view of FIG. 13, the slider actuator assembly 508 comprises a slider body 530 slidably disposed within the main body of the holder 502 and having an upper track 532 and a lower track 534 coupled together via a generally cylindrical body that defines an interior space including a receptacle 538 configured to at least partially receive the removable cartridge, a slider button 540 engaged with the upper track 532 of the slider body 530 so as to move the actuator assembly 508 between a loading position (see FIG. 14A), a lighting position (see FIG. 14B), and an ejecting position (see FIG. 14C). The slider body 530 is configured to slide along a length of the main body in a first direction and a second direction. The holder 502 defines a slot 516 through a surface thereof that is sized and shaped to slidably receive the slider button 540 therein. In some implementations, the upper track 532 of the slider body 530 is sealingly engaged with the holder about the slot 516. In some implementations, the device 500 includes an additional button 540′ disposed on a side of the holder 502 and configured to ignite the ignitable heat source 520 of the cartridge 506. A locating feature 537 (such as, for, example, a spring-loaded ball plunger mechanism) and one or more corresponding detents may be included to temporarily locate the actuator assembly 508 in one or more of the loading, lighting, and ejecting positions.

In an alternative implementation, the slider actuator assembly 508 is configured as a dual-purpose actuator assembly, where the slider button 540 is a spring-loaded push button 540 configured to engage an electrical contact when in the lighting position so as to complete the electrical circuit and deliver electrical current to the ignitor contacts 528 when pressed. See FIG. 14B.

The slider actuator assembly 508 further comprises a pair of static ignitor contacts 528 disposed proximate the distal end 502a of the holder and configured to be engaged with the ignitable heat source when the removable cartridge is secured within the receiving chamber 510. The ignitor contacts 528 are coupled to the printed circuit board 518 via a pair of contact arms 526 that allow the contacts 528 to flex outwardly via insertion of the cartridge 506 so that they contact the ignitable heat source 520 of the cartridge after loading.

The receiving chamber 510 of the device 500 is further defined by an end cap 514 engaged with the distal end 502b of the holder and defining an opening 515 configured to receive the removable cartridge 506 therethrough, a heat sink 586 disposed in the distal end of the holder 502 and secured to the end cap 514 or the holder 502, and an inner slider seal body 546 disposed within the receptacle 538 of the slider body 530 and coupled thereto (e.g., frictionally engaged therewith via a sealing surface). The inner slider seal body 546 defines a cavity configured to sealingly engage and removably secure the removable cartridge 506 therein. For example, the inner slider seal body 546 may include an elastomeric sleeve 548 disposed therein that includes a protuberance or ring 576 extending radially inwardly from a wall of the sleeve that is configured to frictionally and/or sealingly engage an outer surface of the removable cartridge 506. Additionally, the elastomeric sleeve 548 includes a protuberance or ring 570 extending radially outwardly from an exterior wall of the sleeve 548 that is configured to frictionally engage with an inner wall of the receptacle 538. The heat sink 586 defines a passageway that forms a portion of the receiving chamber 510 configured to pass the removable cartridge therethrough and to accommodate the ignitor contacts 528 positioned therein to engage the ignitable heat source 520. The heat sink 586 may be snap fit to the end cap 514 or the distal end 502b of the holder.

The slider body 530 includes an inner stem 533 disposed within the receptacle 538 that is configured to engage one end of the removable cartridge 506 and define a passageway 550 therethrough for passing an aerosol generated from the removable cartridge to the mouthpiece 504. The end cap 514 removably engages the holder 502 via, for example, one or more of a snap-fit, interference fit, screw thread, magnetic, and/or bayonet connection. In other implementations, the end cap may be permanently engaged with the holder 502 after assembly to secure the various components therein. The end cap 514 may be translucent or transparent so as to allow light from the ignitable heat source to pass therethrough when lit. Similarly, the slider button 540 may also be translucent or transparent to allow, for example, light from an LED to pass therethrough that is indicative of a state of the device 500.

The operation of the dual-purpose slider actuator assembly 508 is described with respect to FIGS. 14A-14C, which illustrate a series of cross-sectional side views of the aerosol delivery device 500 in various states of operation. Specifically, FIG. 14A represents the cartridge 506 being loaded into the device 500 (i.e., the actuator assembly in the loading position), FIG. 14B represents the device 500 during ignition and smoking (i.e., the actuator assembly in the ignition position), and FIG. 14C represents the cartridge 506 being ejected from the device 500 (i.e., the actuator assembly in the ejection position).

Referring to FIG. 14A, the cartridge 506 is depicted being inserted in the receiving chamber 510 of the holder 502, so as to locate the cartridge 506 into a lighting and/or use positon. In the depicted implementation, the inner slider seal body 546 forms a cartridge retention assembly configured to retain the cartridge in the receiving chamber in the lighting/use position. As previously described, the inner slider seal body 546 includes an elastomeric sleeve 548 partially disposed on an inner surface of the seal body that includes structure 576 for engaging the cartridge 506. As shown, the seal body has a generally cylindrical shape; however, the shape and size of the seal body 546 may vary to suit a particular application. In particular, the sleeve 548 comprises a silicone seal configured to frictionally engage the cartridge so that when the cartridge 506 is pushed into and fully received within the receiving chamber 510, the cartridge 506 is temporarily “locked” in place within the holder 502. In some implementations, a protuberance 576, such as a ring that extends radially inward from an interior surface of the sleeve 548, is included and is configured to frictionally and/or sealingly engage an outer surface of the removable cartridge 506 to further secure the cartridge within the receiving chamber 510.

In other implementations, other retaining features may be used. For example, in some implementations one or more retention spheres may form part of a cartridge retention assembly. In other implementations, a cartridge retention assembly may comprise one or more resilient members, such as, for example, one or more O-rings, and/or other retaining features that include one or more resilient features that extend into the receiving chamber in order to engage a portion of the outer surface of the cartridge. In other implementations, an outer housing of the cartridge and/or the receiving chamber may include one or more protrusions and/or spring features and corresponding detent features configured to retain the cartridge in the receiving chamber (e.g., a spring-loaded latching mechanism). In still other implementations, an inner surface of the receiving chamber may have a decreasing diameter (and/or one or more portions having a decreased diameter) that may be configured to retain the cartridge in the receiving chamber. In other implementations, the holder may include actively retractable features (e.g., features that are actively retractable by a user) configured to engage the cartridge to retain it in the receiving chamber. In other implementations, the holder may include one or more wedge features configured to engage and retain the cartridge in the receiving chamber. In still other implementations, one or more other features of the cartridge and/or one or more features of the holder may create a releasable connection between the receiving chamber and the cartridge. For example, in some implementations, the cartridge and the receiving chamber may have a releasable screw-type connection. In still other implementations, the cartridge may be retained in the receiving chamber via magnetic force. For example, in some implementations the outer housing of the cartridge may be made of a ferromagnetic material, and the receiving chamber may include one or more magnets. Combinations of two or more of these retaining features may also be used.

In various implementations, one or more components of a cartridge retention assembly may be made of any material, including for example, but not limited to, metal or plastic materials. For example, some implementations may include one or more components of a cartridge retention assembly that are made of a metal material such as, for example, stainless steel, aluminum, brass, copper, silver, gold, bronze, titanium, various alloys, etc. In some implementations, one or more components of a cartridge retention assembly may be made of a moldable plastic material such as, for example, polycarbonate, polyethylene, acrylonitrile butadiene styrene (ABS), polyamide (Nylon), or polypropylene. In some implementations, one or more components of a cartridge retention assembly may be made of a different material, such as, for example, a different plastic material, a different metal material, a graphite material, a glass material, a ceramic material, a natural material (such as, but not limited to, a wood material), a composite material, or any combinations thereof.

As shown in FIG. 14A, the actuator assembly 508 is in a forward orientation (i.e., loading position) within the holder 502. With the actuator assembly 508 in the loading position, the cartridge 506 is manually inserted into the receiving chamber 510 until secured therein with a portion of the cartridge 506 (e.g., the ignitable heat source 520) extending beyond the distal end 502b of the holder 502. In some implementations, the cartridge 506 extends beyond the distal end 502b by about 5 mm to about 25 mm, preferably about 10 mm to about 20 mm, and more preferably about 14.5 mm; however, the cartridge is secured within the inner slider seal body to prevent it from falling out of the receiving chamber 510. In some implementations, the retention structure may include audible or tactile feedback to inform a user that the cartridge 506 is secured within the device 500.

Referring to FIG. 14B, the slider button 540 (and actuator assembly 508) is moved towards the mouthpiece 504 to pull the cartridge 506 into the lighting or use position within the device 500. As shown in FIG. 14B, a proximal end of the cartridge 506 is sealingly engaged with the stem 533 of the slider body 530 and in fluid communication with the hollow stem 505 that extends from the second end 504b of the mouthpiece so as to at least partially define the aerosol passageway 550. In the lighting/use position, the distal end of the cartridge 506 is located proximate the distal end 502b of the holder such that the entire cartridge 506 is located inside of the holder 502. In particular, in the lighting/use position of the depicted implementation, the heat source 520 portion of the cartridge 506 is also positioned proximate the distal end of the holder 502 and aligned with the ignitor contacts 528 in the lighting position. The ignitor contacts 528 remain in contact with the heat source 520 during smoking and until ejected from the device 500. In the alternative implementation with a dual-purpose slider actuator, the slider button 540 is aligned with an electrical contact so as to complete the electrical circuit and deliver electrical current to the ignitor contacts 528 when pressed. In other implementations, the device 500 includes an additional or alternative ignition button 540′ disposed on a side of the holder 502 (see FIGS. 12A and 13).

In some implementations, the device 500 may include an ejection mechanism as shown in FIG. 14C. Generally, the ejection mechanism is configured to eject the cartridge 506 from the distal end 502b of the holder. In one implementation, the ejection mechanism is incorporated in to the actuator assembly 508 so that when the slider push button 540 (and actuator assembly 508) is moved forward until it engages one end of the cartridge 506 and pushes the cartridge out of the receiving chamber 510 and out of the aerosol delivery device 500 through the opening 515 in the end cap 514. Specifically, a user can slide the actuator assembly 508 forward (e.g., using their thumb and the slider button 540) so that the inner stem 533 of the slider body 530 engages the cartridge 506 pushing it out of the inner slider seal body 546 so that the cartridge becomes disengaged from the cartridge sealing structure 576 disposed within the inner slider seal body 546. After which, the cartridge 506 may be pulled out of or allowed to drop out of the receiving chamber 510.

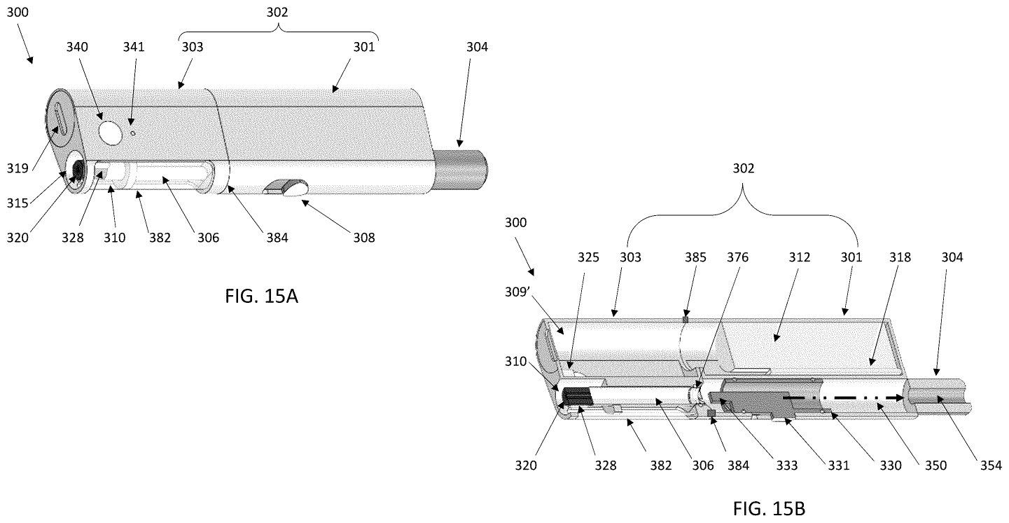

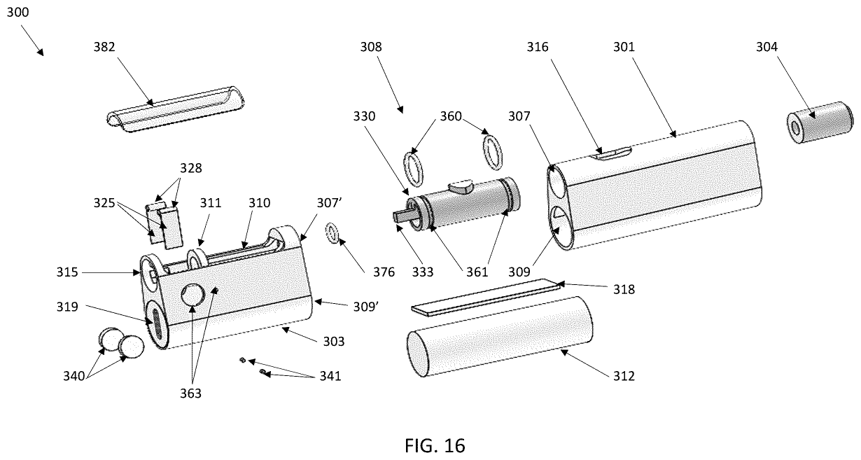

FIGS. 15A, 15B, and 16 illustrate another example implementation of an aerosol delivery device 300. In particular, FIGS. 15A and 8B illustrate a perspective view and a cross-sectional perspective view, respectively, of an aerosol delivery device 300 that includes a removable cartridge 306, a slider assembly 308, and a separate ignition actuator 340. The aerosol delivery device 300 includes a two-part holder 302 comprising an upper body portion 301 and a lower body portion 303, each defining a proximal end and a distal end. However, in alternative implementations, the holder 302 may comprise a single body.

The lower body portion 303 defines a receiving chamber 310 disposed therein and configured to receive a removable cartridge 306 comprising an ignitable heat source 320 and a substrate portion 322 that includes a substrate material having an aerosol precursor composition configured to form an aerosol upon application of heat thereto. The lower body portion 303 includes a pair of actuatable ignitor contacts 328 disposed proximate the distal end thereof and coupled thereto via a pair of elongate bodies 325. The actuatable ignitor contacts 328 are configured to be engageable with the ignitable heat source 320 when the removable cartridge 306 is secured within the receiving chamber 310. Specifically, the ignitor contacts 328 are configured to be movable between a first position spaced apart from the ignitable heat source 320 and a second position contacting the ignitable heat source 320.

In the depicted implementation, each contact 328 and elongate body 325 are formed from a single flat strip of metal (e.g., copper, silver, gold, or other conductive material) where one end is curled or otherwise shaped to form the ignitor contact 328, while the other end remains substantially flat to form the contact arm 328. Alternatively, the contacts 328 and elongate bodies 325 may be formed from a single strip of material, where each end is curled or otherwise shaped to form the first and second ignitor contacts 328 and the middle portion is bent or otherwise shaped to at least partially surround the receiving chamber 310. The middle portion forms a single elongate body 325 that extends on each side of the receiving chamber 310, such that the ignitor contacts 328 may be deflected towards one another and into contact with the ignitable heat source 320. The bend in the single elongate body 325 provides a spring force to bias the ignitor contacts into their first position.

Additionally, the lower body portion 303 includes a window 382 disposed therein and configured to provide a view of at least a portion of the receiving chamber 310 so that a user may observe the cartridge 308 and/or electrical contacts 328. All or a portion of the window may comprise a transparent or translucent material (e.g., a glass material, a polycarbonate, polyethylene terephthalate, acrylic, or the like). In some implementations, a portion of the lower body portion 303 may be made from a transparent or translucent material.

The upper body portion 301 houses a power source 312, a printed circuit board 318 with associated electronics, and the sliding actuator assembly 308. The sliding actuator assembly 308 is slidably disposed within the upper body portion 301 and is configured to eject the removable cartridge from the lower body portion 303. The upper body portion 301 further defines a passageway (e.g., a first aerosol passageway) 350. The upper body portion 301 and the lower body portion 303 are movably coupled together via mechanism 384 (e.g., a swivel joint). So as to enable the lower body portion 303 to rotate relative to the upper body portion 301 to expose the receiving chamber 310 and load the removable cartridge therein. In some implementations, the aerosol delivery device 300 further comprises a locking mechanism 385 disposed on at least one of the lower body portion 303 or the upper body portion 301 that is configured to maintain the device in a closed orientation (i.e., the upper and lower body portions aligned and in fluid and/or electrical communication. The locking mechanism 385 may include, for example, one or more of magnets, friction, a snap fit, or a detent. However, in some other implementations, the upper body portion 301 and the lower body portion 303 are integrally formed together, or may be fixedly coupled with one another. Other manners of movably coupling the upper body portion 301 and the lower body portion 303 include a hinge joint, a sliding track, and the like.

The device 300 further comprises a mouthpiece 304 having a first end 304a and a longitudinally opposed second end 304b with a second aerosol passageway 354 extending longitudinally therebetween. In the depicted implementation, the mouthpiece 304 is located at the proximal end of the upper body portion 301, with the first end configured to engage with a user's mouth and the second end configured to engage the proximal end of the upper body portion 303. The mouthpiece 304 is configured to sealingly engage with the upper body portion 301 so that the first and second aerosol passageways 350, 354 are in fluid communication so as to deliver the aerosol generated in the receiving chamber to the user. The mouthpiece 304 may be removably coupled to the upper body portion 301 to, for example, provide for cleaning or customization of the device 300. However, in other implementations, the mouthpiece 304 may be integrally formed with the upper body portion 301.

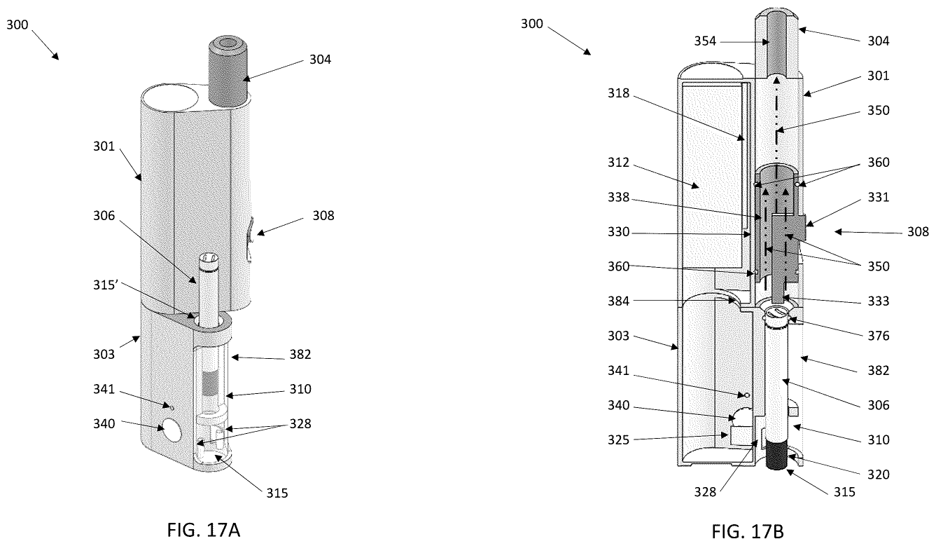

As further illustrated by the exploded view of FIG. 16, the slider actuator assembly 308 comprises a generally tubular slider body 330 disposable within the upper body portion 301 so as to slide along a length of the upper body portion 301 in a first direction and a second direction. The slider body 330 defines a recess 338 extending therethrough that forms a portion of the first aerosol passageway 350 through the upper body portion 301. The slider body 330 includes a first protrusion 331 extending from an outer surface thereof and extending through an opening 316 in a wall of the upper body portion 301 that is configured to move the slider body 330 between a loading position (see FIG. 17A) and an ejecting position (see FIGS. 17C and 17D). The slider body 330 further includes a second protrusion or stem 333 extending from a distal end thereof. The stem 333 is configured to engage the removable cartridge 306 so as to advance the removable cartridge through the distal end of the lower body portion 303 when the actuator assembly 308 is moved into the ejecting position. Portions of the protrusions extend into the receptacle 338 such that the vapor/air path splits around those portions.

The upper body portion 301 further defines two cavities 307, 309, where the first cavity 307 is configured to receive the slider body 130 and the second cavity is configured to house the power source 312 and the printed circuit board 318. Additionally, the actuator assembly 308, specifically the slider body 330, includes a sealing arrangement for sealingly engaging with an internal surface of the first cavity 307 (e.g., to avoid aerosol leakage or undesirable air ingress). In the depicted implementation, the sealing arrangement comprises a pair of O-rings 360 disposed within grooves 361 disposed proximate the distal and proximal ends of the slider body 330. However, other configurations of the sealing arrangement are contemplated and considered within the scope of the disclosure.

The lower body portion 303 also defines two cavities 307′ (includes receiving chamber 310), 309′ that are generally aligned with the cavities 307, 309 of the upper body portion 301. Specifically, the first cavity 307′ is defined by the receiving chamber 310 and opposing openings 315, 315′ and in some implementations includes a bar 311 or other structure for supporting the window 382 and/or guiding the cartridge 306 during insertion. In the depicted implementation, the lower body portion 303 defines a cut-out or other opening that exposes the receiving chamber 310 and is configured to receive the window 382 therein. The window 382 may be installed or otherwise coupled to the lower body portion 303 via an adhesive, snap fit, or other means known in the art. The cavity 307′/receiving chamber 310 includes a groove or other structure for securing a retention mechanism 376 therein that engages with an outer surface of the cartridge 306 as described herein. In the depicted implementation, the retention mechanism 376 is an O-ring.

The second cavity 309′ is generally configured to house various electronics and other mechanisms necessary to provide power to the ignitor contacts 328 and actuate same for igniting the heat source 320 of the cartridge 306 (see FIG. 17B). For example, the elongate bodies 325 are electrically coupled to the power source within the second cavity 309′ and extend through a wall of the second cavity 309′ so as position the ignitor contacts 328 within the receiving chamber 310. The lower body portion 303 further defines a set of openings 363 therethrough that are configured to engage with the actuator buttons 340 and light emitting diodes (LED) 341 that are disposed on both sides of the lower body portion 303 and are described below.

The operation of the device 300 generally, and the slider actuator assembly 308 specifically, is described with respect to FIGS. 17A-17D, which illustrate a series of views of the aerosol delivery device 300 in various states of operation. Specifically, FIG. 17A represents the cartridge 306 being loaded into the device 300 (i.e., the actuator assembly in the loading position), FIG. 17B represents the device 300 during ignition and smoking, and FIGS. 17C and 17D represent the cartridge 306 being ejected from the device 300 (i.e., the actuator assembly in the ejection position).

Referring to FIG. 17A, in order to load the cartridge into the receiving chamber 310, the lower body portion 303 is rotated or otherwise offset (e.g., twisted) from the upper body portion 301 so as to expose the receiving chamber via opening 315′. Prior to rotating the body portions, the actuator assembly 308 is moved into the loading position (i.e., fully retracted towards the mouthpiece 304) so that the sliding body 330 is fully disposed within the first cavity 307 of the upper body portion 301. The cartridge 306 is manually inserted in the receiving chamber 310 of the lower body portion 303, so as to locate the cartridge 306 into a lighting and/or use positon, where the cartridge is sealingly secured within the chamber 310 via the retention mechanism 376 so that the ignitable heat source 320 operatively aligns with the ignitor contacts 328. After loading, the upper and lower body portions are moved back into alignment so that the sealed end of the cartridge 306 is in fluid communication with the first aerosol passageway 350. The fit between the upper and lower body portions is such that there is no aerosol leakage or air ingress therebetween, with or without the use of additional sealing arrangements.

As disclosed above, in some implementations, the holder 302 is a unitary body (e.g., the upper body portion extends essentially the entire length of the device) that is configured to house all of the components as described above, without the need for the rotation and locking mechanisms 384, 385. In such an implementation, the cartridge 306 may be loaded via the distal end of the holder 302 similar to the other implementations described herein.

With reference to FIGS. 15A, 16, 17A, and 17B, in the lighting/use position, the distal end of the cartridge 306 is located proximate the distal end of the lower body portion 303 such that the entire cartridge 306 is located inside of the receiving chamber 310. In particular, in the lighting/use position of the depicted implementation, the heat source 320 portion of the cartridge 306 is also positioned proximate the distal end of the lower body portion 303 and aligned with the ignitor contacts 328 in the lighting position. The heat source 320 is ignited via the aligned push buttons 340 disposed on the opposite sides of the lower body portion 303 of the aerosol delivery device 300. The buttons 340 are movably coupled to the lower body portion, with or without a sealing arrangement, so that a user may press both buttons simultaneously (e.g., via a pinching action) so as to engage and deflect the elongate bodies 325 and by extension move the ignitor contacts 328 into contact with the ignitable heat source 320. Once released, the buttons 340 return to their original position, at least in part via a spring action from the deflected elongate bodies 325 and/or a return biasing element. The device 300 includes a pair of LEDs 341 that may illuminate during ignition and/or change colors to indicate a state of the device 300. Additionally, a user may be able to observe ignition via the window 382.

In some implementations, the device 300 may include an ejection mechanism as shown in FIGS. 17C and 17D. Generally, the ejection mechanism is configured to eject the cartridge 306 from the distal end of lower body portion 303 via opening 315. Between loading and ejection, the cartridge 306 passes through the second cavity 309. In one implementation, the ejection mechanism is incorporated in to the actuator assembly 308 so that when the slider protrusion 331 (and actuator assembly 308) is moved forward until it engages one end of the cartridge 306 and pushes the cartridge out of the receiving chamber 310 and out of the aerosol delivery device 300 through the opening 315 in the lower body portion 303. Specifically, a user can slide the actuator assembly 308 forward (i.e., away from the mouthpiece) using, for example, their thumb and the protrusion 331 so that the inner stem 333 of the slider body 330 engages the cartridge 306 pushing it forward and disengaging the cartridge from the retention mechanism 376. After which, the cartridge 306 may be pulled out of or allowed to drop out of the device 300.

FIG. 17C depicts the device 300 prior to ejection, where the actuator assembly 306 is fully retracted and the slider body 330 fully disposed within the cavity 309. As shown, the proximal end of the cartridge 306 engages the retention mechanism 376, which is configured to retain the cartridge in the receiving chamber. In some implementations, the mechanism 376 is a silicone seal configured to frictionally and/or sealingly engage the cartridge so that when the cartridge 306 is pushed into and fully received within the receiving chamber 310, the cartridge 306 is temporarily “locked” in place. However, in other implementations, the cartridge may include a mating structure (e.g., a depression or groove) configured to engage with the retention mechanism 376 to further secure the cartridge within the receiving chamber 310.

As shown in FIG. 17D, the actuator assembly 308 is moved forward into the ejecting position (i.e., away from the mouthpiece) so that a portion of the stem 333 enters into the cavity 307′ of the lower body portion 303 and engages the cartridge 306. As the actuator assembly is moved forward, the stem 333 pushes the cartridge forward so that the cartridge 306 becomes disengaged from the cartridge retention mechanism 376.

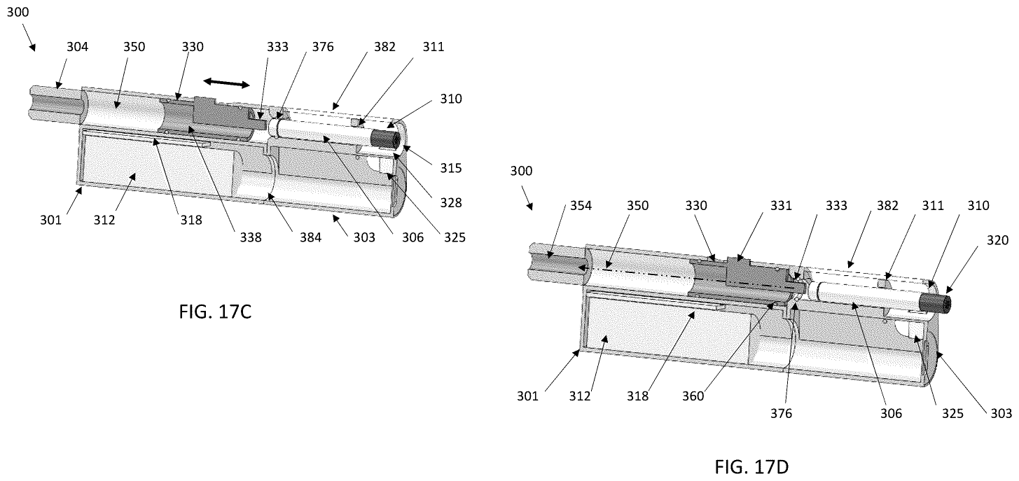

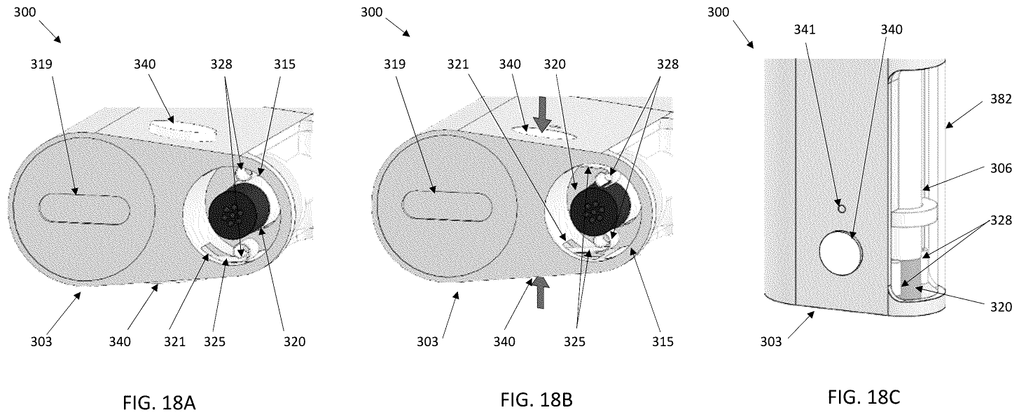

FIGS. 18A and 18B depict distal end views of the aerosol delivery device 300 that further illustrate actuation of the ignitor contacts 328. Specifically, FIG. 18A depicts the ignitor contacts 328 in a first, unactuated position where the ignitor contacts 328 are positioned near, but not in contact with, the ignitable heat source 320. As can be seen through the opening 315 in the distal end of the lower body portion 303 (generally, the holder), there are two openings 321 (only one is shown) provided between the cavities 307′, 309′ (see FIG. 19) through which the elongate bodies 325 extend. Two spring loaded buttons 340 are disposed on opposing sides of the holder 303 and each extends slightly above an outer surface of the holder. The buttons 340 are oriented proximate the elongate bodies 325 (see FIG. 17B) and configured contact the elongate bodies 325 when pressed. Also depicted is the charging port 319.

FIG. 18B depicts the ignitor contacts 328 in a second, actuated position where the contacts 328 are in contact with the ignitable heat source 320. As shown, the spring loaded push buttons 340 are simultaneously pressed to activate ignition. Specifically, when the buttons 340 are pressed, power is activated and the ignitor contacts 328 are pushed onto the ignitable heat source 320. Releasing the buttons 340 terminates power and the ignitor contacts pull away from tip automatically (e.g., return to their first position). FIG. 18C is a partial side view of the distal end of the device 300. As can be seen, there is an LED 341 disposed proximate the push button 340 on each side of the holder 303. The LEDs 341 on both sides illuminate when the buttons 340 are pressed, although in other implementations, the LEDs may illuminate and/or change colors to indicate a status of the device. In addition, a user can see the tip (ignitable heat source 320) of the cartridge 306 ignite through the optional window 382.

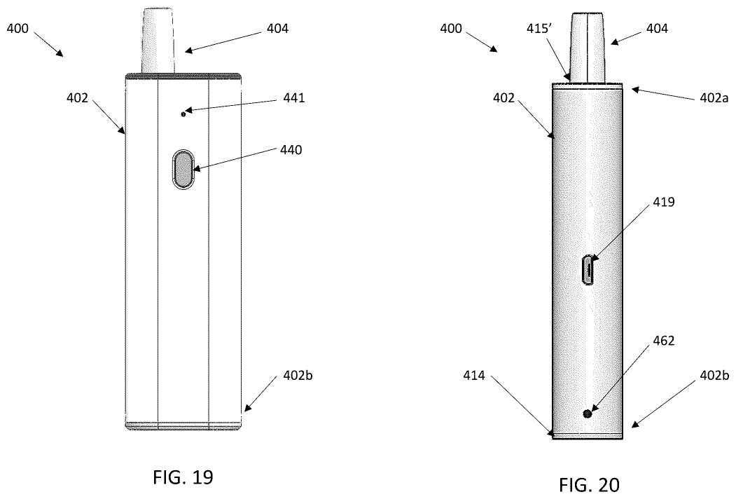

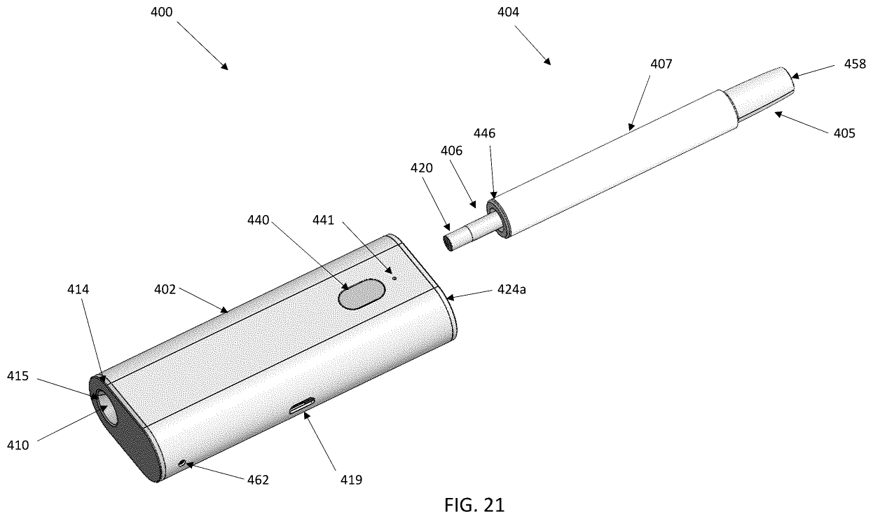

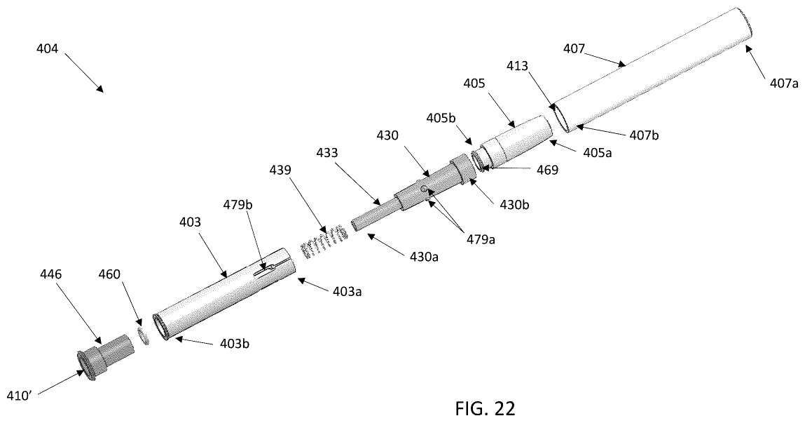

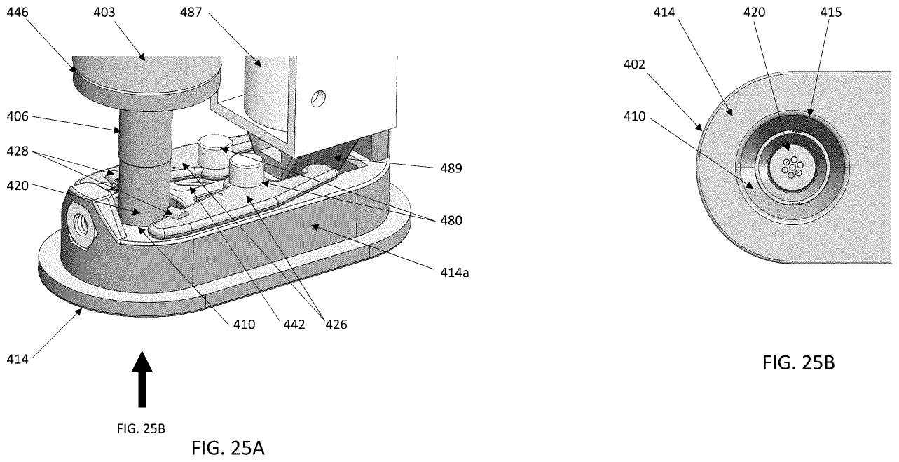

FIGS. 19-27C illustrate another example implementation of an aerosol delivery device 400. In particular, FIGS. 19 and 20 illustrate a front view and a side view, respectively, of the aerosol delivery device 400. In the depicted implementation, the aerosol delivery device 400 includes a holder 402 having a main body defining a proximal end 402a and a distal end 402b, where the holder 402 further defines a receiving chamber 410 configured to receive a removable cartridge 406 (see FIG. 21). In some implementations, the removable cartridge 406 comprises an ignitable heat source 420 and a substrate portion 422 that includes a substrate material having an aerosol precursor composition configured to form an aerosol upon application of heat thereto. The device 400 also includes a pair of actuatable ignitor contacts 428 that are configured to be engageable with the ignitable heat source 420 when the removable cartridge 406 is secured within the receiving chamber 410. Specifically, the ignitor contacts 428 are configured to be movable between a first position spaced apart from the ignitable heat source 420 and a second position contacting the ignitable heat source 420. In some implementations, the holder 402 includes a first biasing mechanism (442 in FIG. 25A) to maintain the ignitor contacts in the first position. The device 400 further includes an ignitor push button 440 configured to ignite the heat source 420 as described below, an LED indicator 441 configured to display a status or operation of the device 400, and a charging port 419 disposed in a sidewall of the device 400.

The device 400 also includes a mouthpiece (also referred to herein as a mouthpiece assembly) 404 removably secured to the holder 402. The removable mouthpiece 404 is configured to engage the proximal end of the holder 402 and may be held therein via a retention mechanism (e.g., mechanical engagement). The mouthpiece 404 is described in greater detail with respect to FIGS. 21-23C. The mouthpiece 404 comprises a first portion 407 comprising an elongate body defined by a first end 407a and a longitudinally opposed second end 407b with a passageway 413 extending therethrough and a second portion 405 defined by a first end 405a and a longitudinally opposed second end 405b with a first aerosol passageway 454 extending therethrough. The first end of the second portion 405 is configured to engage with a user's mouth and a portion of the second end 405b is slidably disposed within the passageway 413 of the first portion 407 of the mouthpiece 404 proximate the first end 407a of the first portion of the mouthpiece 404.

The mouthpiece assembly 404 further includes a slider body 430 defined by a first end 430a and a longitudinally opposed second end 430b with a second aerosol passageway 450 extending therethrough. The slider body 430 is slidably disposed within the first portion of the mouthpiece 407 and the first end 430a of the slider body 430 is coupled to the second end 405b of the second portion 405 of the mouthpiece 404 (e.g., via a retention mechanism 469 similar to any of those disclosed herein) and the second end 430b of the slider body 430 is configured to engage the removable cartridge 406. The second portion 405 of the mouthpiece 404 is configured to sealingly engage with the slider body 430 so that the first and second aerosol passageways 450, 454 are in fluid communication so as to deliver the aerosol generated in the receiving chamber to the user via outlet 458. In the depicted implementation, the second end 430b of the slider body comprises a stem 433 configured to engage with and eject the removable cartridge 406 upon application of a force to the second portion 405 of the mouthpiece. The slider body 430 is also configured to slidably engage an inner tubular body 403 via, for example, one or more projections 479a disposed about an outer surface of the slider body 430 and mating with one or more slots 479b disposed in a wall of the inner tubular body 403. The slider body 430 may be configured to slide along a length of the first portion 407 of the mouthpiece 404 in a first direction and a second direction.

The inner tubular body 403 is defined by a first end 403a and a longitudinally opposed second end 403b and configured to slidably receive the slider body 430 therein. The inner tubular body 403 further defines a receptacle 438 therein that is sized and shaped to retain a second biasing mechanism 439 therein. The second end 403b of the inner tubular body 403 is configured to engage the first portion 407 of the mouthpiece proximate the second end 407b thereof. For example, the inner tubular body 403 may be configured to engage an inner surface of the first portion 407 of the mouthpiece 404 via at least one of complementary-threaded surfaces for a screw-type engagement, a press-fit engagement, a snap-fit engagement, or a magnetic engagement. A portion 410′ of the receiving chamber 410 of the device 400 is at least partially defined by an outlet guide 446 coupled to the second end 407b of the first portion of the mouthpiece 404 and partially received within the second end 403b of the inner tubular body 403. The outlet guide 446 defines the portion of the cavity 410′ that is configured to sealingly engage and removably secure the removable cartridge 406 therein via a retention mechanism (e.g., an O-ring 460) disposed therein.

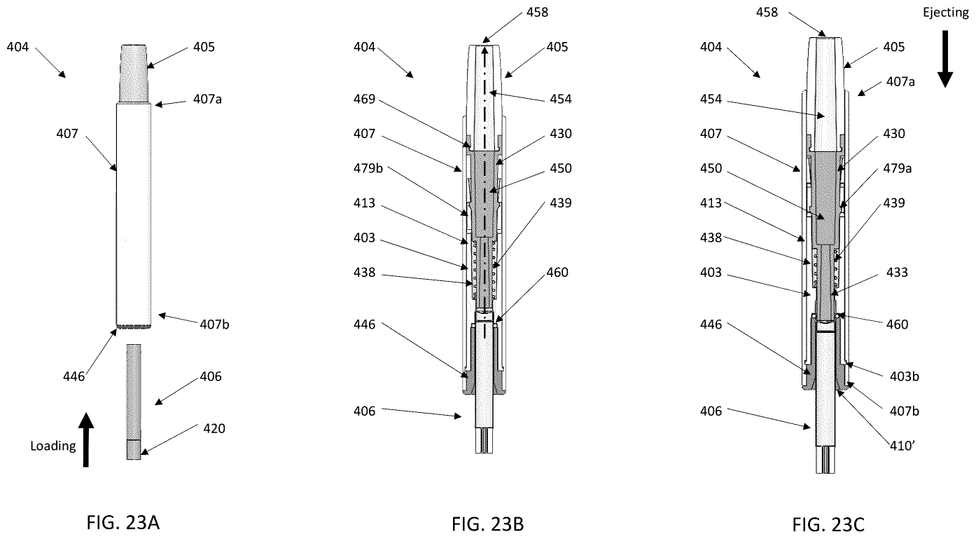

The second biasing mechanism 439 is configured to maintain the mouthpiece assembly 404 in a loading configuration. Specifically, the slider body 430 is biased towards the first end 407a of the first portion 407 of the mouthpiece 404 so that the cartridge may be loaded into and secured within the outlet guide 446 (see FIGS. 23A and 23B). To eject the cartridge 406 from the mouthpiece assembly 404, a user applies a force to (i.e., pushes down on) the second portion 405 of the mouthpiece assembly, which pushes the slider body 430 against the second biasing mechanism 439. The second biasing mechanism 439 is compressed within the inner tubular body 403, so that the stem 433 engages the cartridge 406 and pushes it out of and beyond the retention mechanism 460 (see FIG. 23C). Once the cartridge 406 is disengaged from the retention mechanism, it is free to slide out of the mouthpiece 404.

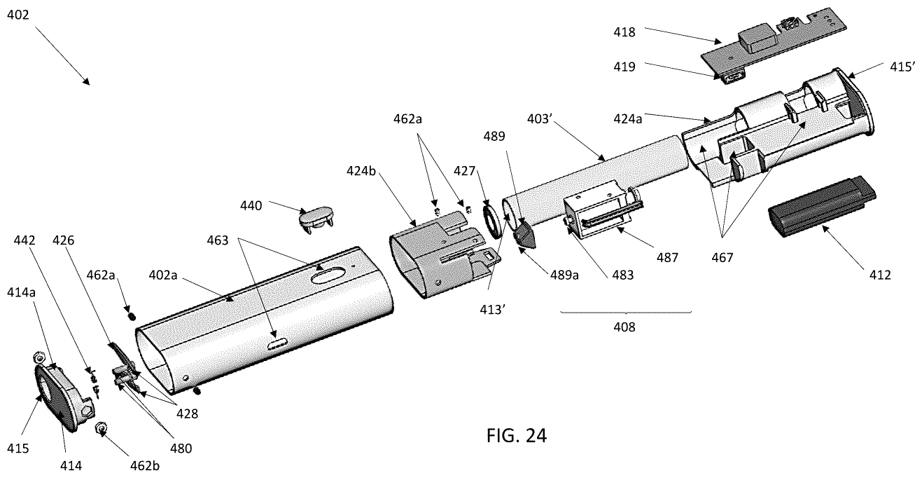

The holder 402 (also referred to herein as the holder assembly) is depicted in greater detail in FIGS. 21 and 24. As illustrated by the exploded view of FIG. 24, the device 400 includes a two-part inner housing 424 disposed within the holder 402 and defining one or more receptacles or cavities 467 defined therein and configured to retain certain components, such as, for example, a power source 412 and/or one or more printed circuit boards 418 therein. The power source 412 is in electrical communication with the printed circuit board 418 and the ignitor contacts 428, which may be energized via the push button 440 disposed on a side wall of the holder 402. Specifically, once the push button 440 is actuated, the electrical circuit is completed and electricity is delivered to the ignitor contacts 428. In some implementations, the ignitor push button 440 may be configured to activate the ignitor contacts 428 for a set time after release or the ignitor contacts will be deactivated when the ignitor push button is released. The printed circuit board 418 may further comprise a charging port 419 that is oriented on the printed circuit board so as to be disposed on the side of the holder 402.

The inner housing 424 comprises and an upper housing 424a and a lower housing 424b that may be coupled together (e.g., via a snap-fit or fasteners) and/or to the holder 402. The holder 402 further comprises an outer tubular body 403′ disposed within a first one of the receptacles 467 and defining an interior cavity or passageway 413′ configured to slidably receive the mouthpiece assembly 404 therein. In the depicted implementation, the passageways 413, 413′ are generally coaxial; however, they need not be coaxial and their orientation may depend on the overall shape and configuration of the device 400. The mouthpiece 404 may be retained therein via magnetic engagement with a ring magnet 427 disposed at a distal end of the outer tubular body 403′. The holder includes an end cap 414 disposed at the distal end 402b of the holder. The end cap 414 is coupled to the distal end 402b of the holder and defines an opening 415 in communication with the interior cavity 413′ of the outer tubular body 403′. The end cap 414 may be configured to secure at least one of the lower (inner) housing 424b or the outer tubular body 403′ within the holder. The upper (inner) housing 424a may include an equivalent structure to the end cap 414 located at its proximal end and configured to secure the upper housing 424a in the holder. In some implementations, the ends of the inner housings 424a, 424b may include retention mechanisms 469 configured to engage with the ends of the holder 402 and/or end cap 414. In one example implementation, the end cap 414 is secured to the holder 402 via mechanical fasteners (e.g., set screws 462a and hex nuts 462b).

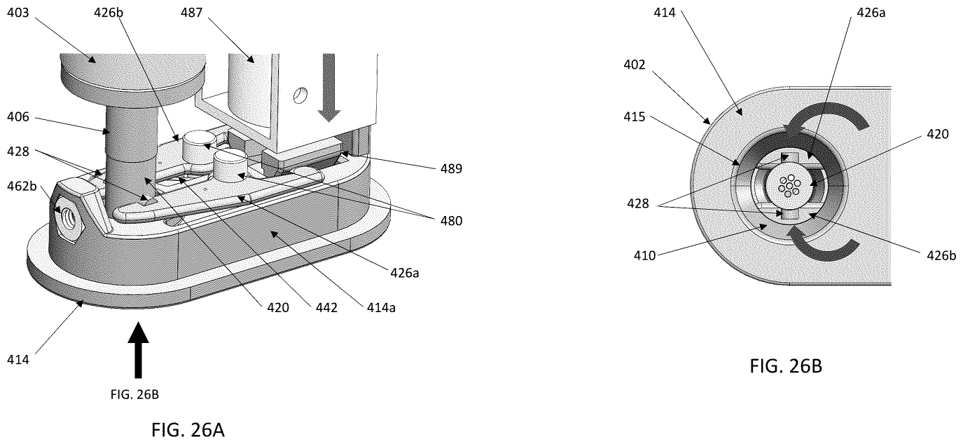

The receiving chamber 410 of the device 400 is further defined by the end cap 414, where the opening 415 is configured to allow air to enter the device 400 and flow through the receiving chamber 410/cartridge 406. The holder 402 further comprises the pair of actuatable ignitor contacts 428 disposed within the receiving chamber 410 proximate the distal end of the holder and movably coupled to the end cap 414 (specifically to a body or stand-off 414a of the end cap 414) via pivot pins 480 and biased into the first position or a neutral configuration (non-ignitable) via the first biasing mechanism 442. The operation of the actuatable ignitor contacts 428 is described in greater detail with respect to FIGS. 25A, 25B, 26A, AND 26B. As shown in FIGS. 24-26B, each of the actuatable ignitor contacts 428 comprises an elongate body 426 with the contact portion 428 disposed proximate a distal end of each of the elongate bodies 426a, 426b. The elongate bodies are pivotably coupled to the end cap 414 disposed within the distal end of the holder 402. The elongate bodies 426 are coupled to the end cap 414 via pivot pins 480 located proximate their midpoints and the first biasing mechanism 442 (e.g., two torsion springs coupled to the proximal ends of the elongate bodies 426) so as to be maintained in the first position. See FIGS. 25A and 25B.

The holder 402 further comprises an actuator assembly 408 disposed therein that is configured to actuate the ignitor contacts 428 between the first position and the second position. The actuator assembly 408 includes a solenoid 487 in electrical communication with the power source 412 and an inclined collar 489 engageable with the solenoid 487 and the proximal end of each of the elongate bodies 426. The actuator assembly 408 is at least partially disposed with one of the receptacles 467 of the inner housing 424 and/or coupled to the outer tubular body 403′. The collar 489 (or other type of driver mechanism) may be coupled to the solenoid 487 via a plunger 483, or may be movably disposed within the lower housing 424b so as to be driven forward or downwardly by the plunger 483 when the solenoid 487 is energized and the plunger 483 advanced. See FIGS. 26A and 26B. In the depicted implementation, the collar 489 has an inclined or wedged shape and is oriented so that a leading edge 489a thereof is inserted between and in contact with the proximal ends of the elongate bodies 426. As the collar 489 is advanced, the inclined surfaces of the collar 489 push the proximal ends of the elongate bodies 426 outwardly so as to pivot the bodies 426 relative to the end cap 414 so that the ignitor contacts 428 are moved into contact with the ignitable heat source 420 of the cartridge 406 (i.e., the second position). When the solenoid 487 is de-energized, the plunger 483 is retracted therein (e.g., spring returned). If the collar 469 is coupled to the plunger 483, it is retracted therewith. Retraction of the collar 489 allows the ignitor contacts 428 to return to the first position via the first biasing mechanism 442. In some implementations, for example where the collar 489 is not coupled to the plunger 483, the spring force of the first biasing mechanism 442 is sufficient to push the collar 489 out from between the proximal ends of the elongate bodies 426 as the plunger 483 is retracted.

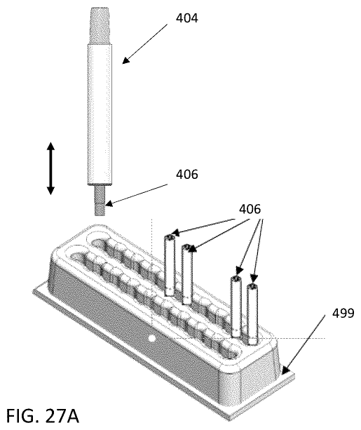

Operation of the aerosol delivery device 400 is further described with respect to FIGS. 27A-27C. Generally, the mouthpiece assembly 404 may have a cartridge 406 loaded therein in a similar manner to aerosol delivery device 100 depicted in FIGS. 4A and 5A. However, in the depicted implementation of FIG. 27A, the mouthpiece 404 may be removed from the holder 402 and brought into contact with a cartridge 406 secured within a package 499 configured to hold multiple cartridges 406. Once the cartridge 406 is loaded into the outlet guide 446, it is retained therein via the retention mechanism 460 and the mouthpiece 404 and the cartridge 406 are withdrawn from the packaging 499. A user can then insert the loaded mouthpiece 404 into the holder 402 via the opening 415′ in the proximal end of the upper housing 424a, as shown in FIG. 27B (see also FIG. 21). The mouthpiece 404 may be retained within the holder 402 via the retention mechanism 427 until the user decides to remove/replace the cartridge (e.g., after the cartridge is fully consumed). The mouthpiece 404 may be removed by an application of a pulling and/or twisting action thereto to overcome the engagement between the mouthpiece 404 and holder 402. After removing the mouthpiece 404 from the holder 402, the cartridge 406 may be ejected from the mouthpiece as described above with respect to FIG. 23C. In some implementations, the mouthpiece 404 does not need to be removed from the holder 402 to eject the cartridge 406. The cartridge is still ejected by pressing down on the second portion 405 of the mouthpiece 404 and pushing the cartridge 406 distally out of the outlet guide 446, but the cartridge continues to travel forward and out of the opening 415 in the end cap 414 of the holder 402.

Once fully inserted, the user can press the ignitor push button 440 to light the ignitable heat source 420 of the cartridge 406. Specifically, pressing the ignitor push button 440 arms or otherwise activates the ignitor contacts 428 (i.e., electrically couples the contacts 428 to the power source 412) as shown in FIG. 27C. Actuating the push button 440 also electrically couples the power source 412 to the actuator assembly, specifically the solenoid 487, which causes the energized ignitor contacts 428 to move in to contact with the ignitable heat source 420 (i.e., the second position).

In some implementations, a LED 441 associated with the push button 440 is illuminated when the ignitor contacts 428 are energized and may change state (e.g., flashing to solid, different colors or intensity, etc.) when the ignitor contacts 428 are brought into contact with the cartridge 406. In various implementations, the device 400 may be programmed so that the ignitor contacts are de-energized after the push button 440 is released and/or after a set period of time (e.g., 30 seconds). Similarly, the solenoid 487 may be energized as long as the button 440 is depressed or for a set period of time, for example, a time sufficient for ignition to be achieved. In some implementations, the actuator assembly 408 (i.e., solenoid 487) may be a “latching” type that maintains its extended configuration without a continuous draw of power. The actuator assembly 408 may be retracted by pushing the button a second time or twice in rapid succession, or similar means. The LED 441 may also be configured to indicate various other states of operation (low power, charging, etc.) by, for example, changing colors, flashing, etc.

In some implementations, the mouthpiece assembly 404 may be removed from the holder 402 after ignition thereof so that the user only needs to hold onto the mouthpiece assembly 404. Alternatively, or additionally, the cartridge 406 may be manually lit so as to eliminate the need for the holder 402 in certain circumstances.

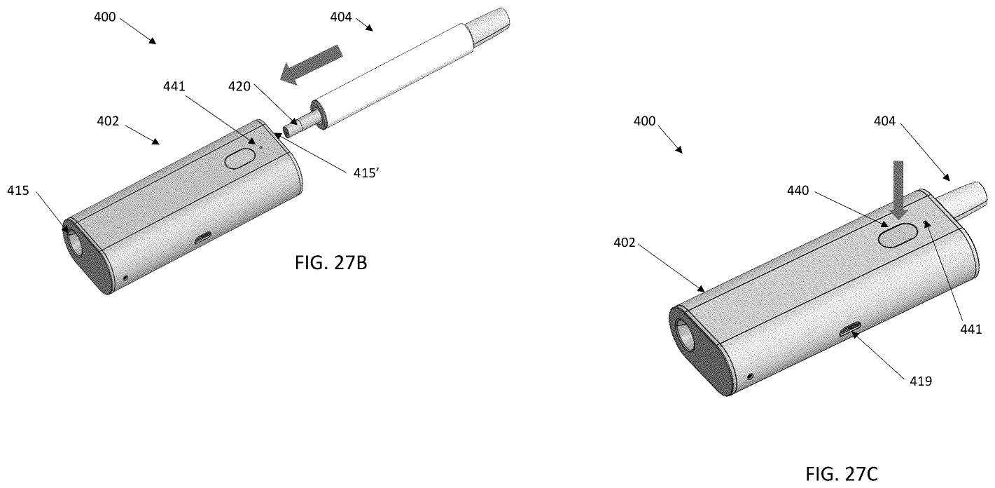

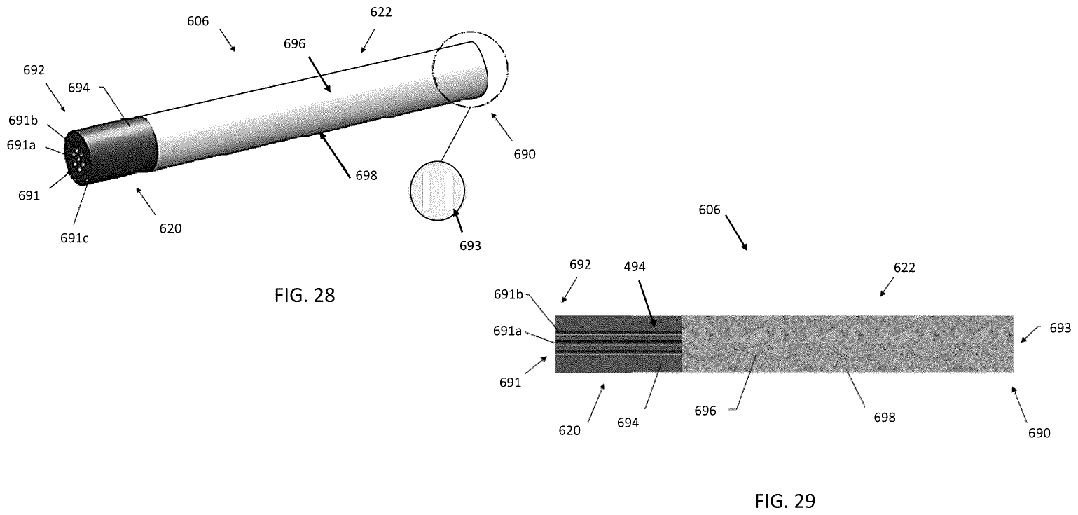

In the depicted implementations, the outer housing or holder 102 may comprise a rigid material. For example, the holders 102, 202, 301, 303, 402, 502 of the depicted implementations may be constructed of an aluminum material; however, in other implementations, the holders may be constructed of other materials, including other metal materials (such as, for example, stainless steel, aluminum, brass, copper, silver, gold, bronze, titanium, various alloys, etc.), or graphite materials, or ceramic materials, or plastic materials, or any combinations thereof. In some implementations, at least a portion of the heat source and/or at least a portion of the substrate material may be circumscribed by a paper foil laminate. In some implementations, the cartridge may comprise an enclosure comprising a laminate that contains a heat source and a beaded substrate material. Some examples of laminates and/or enclosures that may be applicable to the present disclosure can be found in U.S. Pat. App. Pub. No. 2020/0128880 to Gage et al., which is incorporated herein by reference in its entirety. Other examples of cartridges are described herein below with respect to FIGS. 28 and 29.

In some implementations, the holder (or any components thereof) may be made of moldable plastic materials such as, for example, polycarbonate, polyethylene, acrylonitrile butadiene styrene (ABS), polyamide (Nylon), or polypropylene. In other implementations, the holder may be made of a different material, such as, for example, a different plastic material, a metal material (such as, but not limited to, stainless steel, aluminum, brass, copper, silver, gold, bronze, titanium, various alloys, etc.), a graphite material, a glass material, a ceramic material, a natural material (such as, but not limited to, a wood material), a composite material, or any combinations thereof. The holders may be formed via extrusion. As noted above, the mouthpiece portion of some implementations is separable from the main body, while in other implementations, the mouthpiece portion may be integral with the main body. In any event, the mouthpiece portion and the main body may be made of the same material or different materials. In various implementations comprising a separable mouthpiece portion, the mouthpiece portion may be coupled to the main body in a variety of ways, including, for example, via one or more of a snap-fit, interference fit, screw thread, magnetic, and/or bayonet connection. In other implementations, the mouthpiece portion may be integral with the main body and thus may not be separable.

In the depicted implementations, the holder includes walls that are substantially solid and non-porous; however, in other implementations one or more of these walls of a holder may have other configurations. For example, in some implementations one or more of the walls of a holder may be non-solid and/or substantially porous or may include one or more non-solid and/or substantially porous portions. In some implementations, for example, the holder may include one or more apertures that may facilitate access of oxygen to the heat source. Alternatively, or additionally, other implementations may include one or more apertures that may mix with the aerosol generated during a draw. In such a manner, in the use position the one or more apertures may be located proximate the heat source, thus providing the heat source with additional access to oxygen during combustion. In some implementations, the holder may include one or more apertures downstream from the heat source. For example, in some implementations the holder may include apertures that extend into the aerosol passage of the holder that may mix with aerosol generated by the substrate material of the cartridge.

As described above, the holder of various implementations of the present disclosure includes a lighting/use position. In some implementations, the holder may also have an extinguishment position. In such a manner, the extinguishment position may be configured such that the heat source of a cartridge is deprived of sufficient oxygen to sustain combustion. In some implementations, the extinguishment position may be obtained by a further action of the holder. In other implementations, one or more additional features may be included such that an extinguishment position may be achieved by actuating the one or more additional features. In particular, the holder of one implementation may include an air impermeable cover feature located proximate the distal end of the holder that may be mechanically or manually actuatable (e.g., by rotating the cover feature over the end of the main body and/or by sliding the cover feature across the end of the main body) such that in the extinguishment position, the cover feature substantially covers the open end of the holder and the heat source of the cartridge is deprived of sufficient oxygen to sustain combustion. In another implementation, the holder may include a detachable feature, such as, for example, an end cap, that may be used to achieve the extinguishment position. For example, in some implementations a separate end cap may be attachable over the distal end of the holder such that, once attached, the heat source of the cartridge is deprived of sufficient oxygen to sustain combustion. Such an end cap could also be used to cover the end of the second body portion when not in use, such as, for example, to prevent dirt and/or foreign objects from entering into the device. Additionally, or alternatively, in some implementations the holder of the present disclosure may include an air permeable cover feature (e.g., a cover feature comprising a plurality of openings or a cover feature comprising a mesh) that protects the heat source of the cartridge in the lighting/use position. For example, the holder of one implementation may include an air permeable cover feature located proximate the distal end of the holder that may be mechanically or manually actuatable (e.g., by rotating the cover feature over the end of the holder and/or by sliding the cover feature across the end of the holder) such that once ignited, the cover feature may be actuated to substantially cover the open end of the holder while maintaining sufficient access of oxygen to the heat source.

In various implementations, a removable cartridge may have other configurations for use with a holder of the present disclosure. For example, FIG. 28 illustrates a perspective view of a removable cartridge 606, according to another example implementation of the present disclosure. In the depicted implementation, the cartridge 606 defines a proximal end 690 and a distal end 692. The cartridge 606 of the depicted implementation further includes an ignitable heat source 620, which comprises a fuel element 694, a substrate portion 622, which comprises a substrate material 696 (see FIG. 29), and an outer housing 698 configured to circumscribe at least a portion of the ignitable heat source 620 and the substrate material 622. It should be noted that although in the depicted implementation the cartridge 606 has a substantially cylindrical overall shape, in various other implementations, the cartridge or any of its components may have a different shape. For example, in some implementations the cartridge (and/or any of its components) may have a substantially rectangular shape, such as a substantially rectangular cuboid shape. In other implementations, the cartridge (and/or any of its components) may have other hand-held shapes. Some examples of cartridge configurations that may be applicable to the present disclosure can be found in U.S. patent application Ser. No. 16/515,637, filed on Jul. 18, 2019, and titled AerosolDelivery Device with Consumable Cartridge, which is incorporated herein by reference in its entirety.

In some implementations, a barrier may exist between the heat source and the substrate material. In some implementations, such a barrier may comprise a disc that may include one or more apertures therethrough. In some implementations, the barrier may be constructed of a metal material (such as, for example, stainless steel, aluminum, brass, copper, silver, gold, bronze, titanium, various alloys, etc.), or a graphite material, or a ceramic material, or a plastic material, or any combinations thereof. In some implementations, a heat transfer component, which may or may not comprise a barrier, may exist between the heat source and the substrate material. Some examples of heat transfer components are described in U.S. Pat. App. Pub. No. 2019/0281891 to Hejazi et al., which is incorporated herein by reference in its entirety. In some implementations, a barrier and/or a heat transfer component may prevent or inhibit combustion gasses from being drawn through the substrate material (and/or from being drawn through air passageways through which aerosol is drawn).

In various implementations, the heat source may be configured to generate heat upon ignition thereof. In the depicted implementation, the ignitable heat source 620 comprises a combustible fuel element 694 that has a generally cylindrical shape and that incorporates a combustible carbonaceous material. In other implementations, the heat source may have a different shape, for example, a prism shape having a cubic or hexagonal cross-section. Carbonaceous materials generally have a high carbon content. Some carbonaceous materials may be composed predominately of carbon, and/or typically have carbon contents of greater than about 60 percent, generally greater than about 70 percent, often greater than about 80 percent, and frequently greater than about 90 percent, on a dry weight basis.

In some instances, the heat source may incorporate elements other than combustible carbonaceous materials (e.g., tobacco components, such as powdered tobaccos or tobacco extracts; flavoring agents; salts, such as sodium chloride, potassium chloride and sodium carbonate; heat stable graphite a hollow cylindrical (e.g., tube) fibers; iron oxide powder; glass filaments; powdered calcium carbonate; alumina granules; ammonia sources, such as ammonia salts; and/or binding agents, such as guar gum, ammonium alginate and sodium alginate). In other implementations, the heat source may comprise a plurality of ignitable objects, such as, for example, a plurality of ignitable beads. It should be noted that in other implementations, the heat source may differ in composition or relative content amounts from those listed above. For example, in some implementations different forms of carbon could be used as a heat source, such as graphite or graphene. In other implementations, the heat source may have increased levels of activated carbon, different porosities of carbon, different amounts of carbon, blends of any above mentioned components, etc. In still other implementations, the heat source may comprise a non-carbon heat source, such as, for example, a combustible liquefied gas configured to generate heat upon ignition thereof. For example, in some implementations, the liquefied gas may comprise one or more of petroleum gas (LPG or LP-gas), propane, propylene, butylenes, butane, isobutene, methyl propane, or n-butane. In still other implementations, the heat source may comprise a chemical reaction based heat source, wherein ignition of the heat source comprises the interaction of two or more individual components. For example, a chemical reaction based heat source may comprise metallic agents and an activating solution, wherein the heat source is activated when the metallic agents and the activating solution come in contact. Some examples of chemical based heat sources can be found in U.S. Pat. No. 7,290,549 to Banerjee et al., which is incorporated herein by reference in its entirety. Combinations of heat sources are also possible. Although specific dimensions of an applicable heat source may vary, in the depicted implementation, the ignitable heat source 620 has a length in an inclusive range of approximately 5 mm to approximately 20 mm, and in some implementations may be approximately 12 mm, and an overall diameter in an inclusive range of approximately 3 mm to approximately 8 mm, and in some implementations may be approximately 4.8 mm (and in some implementations, approximately 7 mm).

Although in other implementations the heat source may be constructed in a variety of ways, in the depicted implementation, the ignitable heat source 620 is extruded or compounded using a ground or powdered carbonaceous material, and has a density that is greater than about 0.5 g/cm³, often greater than about 0.7 g/cm³, and frequently greater than about 1 g/cm³, on a dry weight basis. See, for example, the types of fuel source components, formulations and designs set forth in U.S. Pat. No. 5,551,451 to Riggs et al. and U.S. Pat. No. 7,836,897 to Borschke et al., which are incorporated herein by reference in their entireties.

In various implementations, the heat source may have a variety of forms, including, for example, a substantially solid cylindrical shape or a hollow cylindrical (e.g., tube) shape. In other implementations, the heat source may comprise a plurality of hollow or substantially solid spheres, which in some implementations may comprise substantially the same size, and in other implementations may comprise more than one size. In various implementations, the heat source may be made in variety of ways, including, but not limited to, via extrusion, injection molding, compression molding, etc. The ignitable heat source 620 of the depicted implementation comprises an extruded monolithic carbonaceous material that has a generally cylindrical shape that includes a plurality of internal passages 691 extending longitudinally from a first end of the ignitable heat source 620 to an opposing second end of the ignitable heat source 620. In the depicted implementation there are approximately thirteen internal passages 691 comprising a single central internal passage 691a, six surrounding internal passages 680b, which are spaced from the central internal passages 691a and have a similar size (e.g., diameter) to that of the central internal passage 691a, and six peripheral internal passages 691c, which are spaced from an outer surface of the ignitable heat source 620 and are smaller in diameter than that of the central internal passage 691a. It should be noted that in other implementations, there need not be a plurality of internal passages and/or the plurality of internal passages may take other forms and/or sizes. For example, in some implementations, there may be as few as two internal passages, and still other implementations may include as few as a single internal passage. Still other implementations may include no internal passages at all. Additional implementations may include multiple internal passages that may be of unequal diameter and/or shape and which may be unequally spaced and/or located within the heat source.

Some implementations may alternatively, or additionally, include one or more peripheral grooves that extend longitudinally from a first end of the heat source to an opposing second end, although in other implementations the grooves need not extend the full length of the heat source. In some implementations, such grooves may be substantially equal in width and depth and may be substantially equally distributed about a circumference of the heat source. In such implementations, there may be as few as two grooves, and still other implementations may include as few as a single groove. Still other implementations may include no grooves at all. Additional implementations may include multiple grooves that may be of unequal width and/or depth, and which may be unequally spaced around a circumference of the heat source. In still other implementations, the heat source may include flutes and/or slits extending longitudinally from a first end of the extruded monolithic carbonaceous material to an opposing second end thereof. In some implementations, the heat source may comprise a foamed carbon monolith formed in a foam process of the type disclosed in U.S. Pat. No. 7,615,184 to Lobovsky, which is incorporated herein by reference in its entirety. As such, some implementations may provide advantages with regard to reduced time taken to ignite the heat source. In some other implementations, the heat source may be co-extruded with a layer of insulation (not shown), thereby reducing manufacturing time and expense. Other implementations of fuel elements include carbon fibers of the type described in U.S. Pat. No. 4,922,901 to Brooks et al. or other heat source implementations such as is disclosed in U.S. Pat. App. Pub. No. 2009/0044818 to Takeuchi et al., each of which is incorporated herein by reference in its entirety. Further examples of heat sources including debossed heat source systems, methods, and smoking articles that include such heat sources are disclosed in U.S. Pat. App. Pub. No. 2019/0254335 to Spicer et al., which is incorporated herein by reference in its entirety.

Generally, the heat source is positioned sufficiently near an aerosol delivery component (e.g., the substrate portion) having one or more aerosolizable components so that the aerosol formed/volatilized by the application of heat from the heat source to the aerosolizable components (as well as any flavorants, medicaments, and/or the like that are likewise provided for delivery to a user) is deliverable to the user by way of the mouthpiece. That is, when the heat source heats the substrate component, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof. Additionally, the selection of various smoking article elements is appreciated upon consideration of commercially available electronic smoking articles, such as those representative products listed in the background art section of the present disclosure.

FIG. 29 illustrates a longitudinal cross-section view of the cartridge 606 of FIG. 28. As shown in the figure, the substrate material 622 of the depicted implementation has opposed first and second ends, with the ignitable heat source 620 disposed adjacent the first end of the substrate material 622. Although dimensions and cross-section shapes of the various components of the cartridge may vary due to the needs of a particular application, in the depicted implementation the cartridge 606 may have an overall length in an inclusive range of approximately 10 mm to approximately 50 mm and a diameter in an inclusive range of approximately 2 mm to approximately 20 mm. In addition, in the depicted implementation the outer housing 698 may have a thickness in the inclusive range of approximately 0.05 mm to 0.5 mm. Furthermore, in the depicted implementation the substrate portion 610 may have a length in the inclusive range of approximately 5 mm to 30 mm and a diameter slightly less than that of the overall cartridge in order to accommodate the thickness of the housing 698, such as, for example, a diameter in an inclusive range of approximately 2.9 mm to approximately 9.9 mm. In the depicted implementation, the substrate material 622 comprises tobacco beads, which may have diameter sizes in range of approximately 0.5 mm to 2.0 mm, although in other implementations the size may differ. In other implementations, the substrate material may be a granulated tobacco material or cut filler tobacco. Although other implementations may differ, in the depicted implementation the outer housing 698 of the cartridge 606 is filled to about 80-90% capacity to allow for insertion of the fuel element 694.

In the depicted implementation, the substrate portion 622 comprises a substrate material 696 having a single segment, although in other implementations the substrate portion may include one or more additional substrate material segments. For example, in some implementations, the aerosol delivery device may further comprise a second substrate material segment (not shown) having opposed first and second ends. As described above, in various implementations, one or more of the substrate materials may include a tobacco or tobacco related material, with an aerosol precursor composition associated therewith. In other implementations, non-tobacco materials may be used, such as a cellulose pulp material. In other implementations, the non-tobacco substrate material may not be a plant-derived material. Other possible compositions and/or components for use in a substrate material (and/or substrate materials) are described above. Reference is also made to the discussion above regarding various possible shapes, aerosol precursor compositions, additives, flavorants, etc. of the substrate material.

As shown in FIGS. 28 and 29, the outer housing 698 of the cartridge 606 of the depicted implementation is configured to circumscribe at least a portion of the substrate portion 622, including the substrate material 696. In the depicted implementation, the outer housing 698 is also configured to circumscribe a portion of the ignitable heat source 620. In some implementations, the outer housing may circumscribe the entire heat source. In the depicted implementation, the outer housing comprises a rigid material. For example, the outer housing 698 of the depicted implementation is constructed of an aluminum material; however, in other implementations the outer housing may be constructed of other materials, including other metal materials (such as, for example, stainless steel, aluminum, brass, copper, silver, gold, bronze, titanium, various alloys, etc.), or graphite materials, or ceramic materials, or plastic materials, or any combinations thereof. In some implementations, at least a portion of the heat source and/or at least a portion of the substrate material may be circumscribed by a paper foil laminate. In some implementations, the cartridge may comprise an enclosure comprising a laminate that contains a heat source and a beaded substrate material. Some examples of laminates and/or enclosures that may be applicable to the present disclosure can be found in U.S. Pat. App. Pub. No. 2020/0128880 to Gage et al., which is incorporated herein by reference in its entirety.

In the depicted implementation, the outer housing 698 is constructed as a tube structure that substantially encapsulates the substrate material 622; however, as noted above, in other implementations the outer housing may have other shapes. Although the shape of the outer housing may vary, in the depicted implementation the outer housing 698 comprises a tube structure having an open end and a closed end. The depicted implementation of the outer housing 698 also includes one or more end apertures 693 located on the closed end of the outer housing 698 that are configured to allow aerosolized vapor (herein alternatively referred to as a “vapor” or “aerosol”) to pass therethrough. The end apertures 693 of the depicted implementation are in the form of a pair of elongate rounded slots; however, in other implementations the end apertures may have any form that permits passage of the aerosol therethrough. As such, it will be appreciated that the end apertures 693 can comprise fewer or additional apertures and/or alternative shapes and sizes of apertures than those illustrated.

In various implementations, the present disclosure may also be directed to kits that provide a variety of components as described herein. For example, a kit may comprise a holder with one or more cartridges. In another implementation, a kit may comprise a plurality of holders. In further implementations, a kit may comprise a plurality of cartridges. In yet another implementation, a kit may comprise a plurality of holders and a plurality of cartridges. The inventive kits may further include a case (or other packaging, carrying, or storage component) that accommodates one or more of the further kit components. The case could be a reusable hard or soft container. Further, the case could be simply a box or other packaging structure. In some implementations, a brush or other cleanout accessory may be included in a kit. The cleanout accessory may be configured to be inserted in a cartridge receiving chamber of the holder, or, in other implementations, inserted in a separate aperture that enables a user to remove debris from the cartridge receiving chamber.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

What is claimed is:

1. An aerosol delivery device comprising:
a holder comprising a main body defining a proximal end and a distal end, the main body further defining a receiving chamber configured to receive a removable cartridge comprising an ignitable heat source and a first aerosol passageway that extends through at least a portion of the main body;
a mouthpiece including a first end and a longitudinally opposed second end with a second aerosol passageway extending longitudinally therebetween, wherein the first end is configured to engage with a user's mouth and the second end is configured to engage the proximal end of the holder;
a power source disposed within the main body; and
a dual-purpose actuator assembly coupled to the holder and configured to ignite the ignitable heat source and eject the removable cartridge.

2. The aerosol delivery device of claim 1, wherein the dual-purpose actuator assembly comprises:
a slider body slidably disposed within the main body of the holder and defining a receptacle configured to at least partially receive the removable cartridge, the slider body configured to slide along a length of the main body in a first direction and a second direction;
a spring-loaded push button coupled to the slider body so as to move the dual-purpose actuator assembly between a loading position, a lighting position, and an ejecting position, the push button configured to engage an electrical contact when in the lighting position; and
a spring assembly configured to engage the slider body and bias the dual-purpose actuator assembly into the loading position.

3. The aerosol delivery device of claim 1, wherein the dual-purpose actuator assembly comprises:
a slider body slidably disposed within the main body of the holder and comprising an upper track, a lower track, and a collar coupling the upper and lower tracks and defining a receptacle configured to at least partially receive the removable cartridge, the slider body configured to slide along a length of the main body in a first direction and a second direction;
a spring-loaded push button engageable with the upper track of the slider body so as to move the dual-purpose actuator assembly between a loading position, a lighting position, and an ejecting position, the push button configured to engage an electrical contact when in the lighting position; and
a spring assembly configured to engage the slider body and bias the dual-purpose actuator assembly into the loading position.

4. The aerosol delivery device of claim 3, wherein the receiving chamber comprises:
an end cap engaged with the distal end of the main body and defining an opening configured to receive the removable cartridge;
an outlet guide coupled to the slider body and slidably disposed within the main body and through the end cap, the outlet guide defining a passageway therethrough configured to pass the removable cartridge therethrough; and
an inner slider seal body disposed within the receptacle of the slider body and sealingly coupled thereto, the inner slider seal body defining a cavity configured to sealingly engage and removably secure the removable cartridge therein.

5. The aerosol delivery device of claim 4 further comprising a pair of actuatable ignitor contacts coupled to the dual-purpose actuator assembly and disposed proximate a distal end of the receiving chamber and configured to be engaged with the ignitable heat source when the removable cartridge is secured within the receiving chamber.

6. The aerosol delivery device of claim 5, wherein the dual-purpose actuator assembly further comprises:
a first contact arm pivotably coupled to the main body and configured to receive one of the pair of actuatable ignitor contacts, the first contact arm actuated via contact with the lower track of the slider body when the dual-purpose actuator assembly is moved into the lighting position; and
a second contact arm pivotably coupled to the main body and configured to receive the other one of the pair of actuatable ignitor contacts, the second contact arm actuated via contact with the lower track of the slider body when the dual-purpose actuator assembly is moved into the lighting position,
wherein actuation of the first and second contact arms moves the ignitor contacts into contact with the ignitable heat source.

7. The aerosol delivery device of claim 3, wherein the holder defines a slot through a surface thereof, wherein the upper track of the slider body is sealingly engaged with the holder about the slot and the slot is configured to slidably receive the spring-loaded push button therein.

8. The aerosol delivery device of claim 3 further comprising:
an inner housing disposed within the main body of the holder; and
a printed circuit board in electrical communication with the power source and comprising the electrical contact, wherein the inner housing is configured to receive the power source and the printed circuit board.

9. The aerosol delivery device of claim 8, wherein the printed circuit board further comprises a charging port and the printed circuit board is oriented within the inner housing so that the charging port is disposed at the proximal end of the holder.

10. The aerosol delivery device of claim 3, wherein the receptacle of the slider body comprises an inner stem engageable with one end of the removable cartridge and defining a passageway therethrough for passing an aerosol generated from the removable cartridge.

11. The aerosol delivery device of claim 5, wherein the outlet guide comprises a pair of opposing slots configured to allow the ignitor contacts to pass therethrough and engage the ignitable heat source.

12. The aerosol delivery device of claim 4, wherein the end cap is translucent and configured to allow light from the ignitable heat source to pass therethrough when lit.

13. The aerosol delivery device of claim 10, wherein the inner stem is configured to engage with and eject the removable cartridge when the dual-purpose actuator assembly is moved to the ejecting position.

14. An aerosol delivery device comprising:
a holder comprising a main body defining a proximal end and a distal end, the main body further defining a receiving chamber configured to receive at least a portion of a removable cartridge comprising an ignitable heat source and a passageway that extends through at least a portion of the main body;
a mouthpiece configured to engage with a user's mouth;
a power source disposed within the main body; and
a pair of actuatable ignitor contacts disposed proximate the distal end of the main body and configured to be engaged with the ignitable heat source when the removable cartridge is secured within the receiving chamber, wherein the ignitor contacts are movably coupled to the holder and movable between a first position spaced apart from the ignitable heat source and a second position contacting the ignitable heat source.

15. The aerosol delivery device of claim 14, wherein each of the actuatable ignitor contacts comprises an elongate body and a contact portion disposed at a distal end thereof, a proximal end of the elongate body pivotably coupled to the main body and wherein the contact portions are pivotable towards each other and into the second position.

16. The aerosol delivery device of claim 14, wherein the pair of actuatable ignitor contacts comprises an elongate body extending between the actuatable ignitor contacts, the elongate body coupled to the main body and shaped to at least partially surround the removable cartridge, such that the actuatable ignitor contacts are deflectable towards each other and into the second position.

17. The aerosol delivery device of claim 14, wherein each of the actuatable ignitor contacts comprises an elongate body and a contact portion disposed at a distal end thereof, the elongate bodies pivotably coupled to the main body and shaped to bias the ignitor contacts into the first position.

18. The aerosol delivery device of claim 14, wherein the actuatable ignitor contacts are in electrical communication with the power source.

19. The aerosol delivery device of claim 14 further comprising an actuator assembly coupled to the holder and configured to move the actuatable ignitor contacts between the first position and the second position.

20. The aerosol delivery device of claim 19, wherein the actuator assembly is configured to couple the actuatable ignitor contacts to the holder.

21. The aerosol delivery device of claim 19, wherein the actuator assembly moves the ignitor contacts into electrical communication with the power source.

22. The aerosol delivery device of claim 19, wherein the actuator assembly comprises:
a slider body slidably disposed within the main body of the holder and defining a receptacle configured to at least partially receive the removable cartridge, the slider body configured to slide along a length of the main body in a first direction and a second direction;
an actuator coupled to the slider body and configured to move the actuator assembly between a loading position, a lighting position, and an ejecting position; and
a spring assembly configured to engage the slider body and bias the actuator assembly into the loading position,
wherein the actuator assembly is configured to engage the actuatable ignitor contacts so as to move the ignitor contacts from the first position to the second position.

23. The aerosol delivery device of claim 22, wherein the actuator assembly further comprises a spring-loaded push button coupled to the slider body and configured to be engageable with an electrical contact when in the lighting position so as to deliver electrical current to the ignitor contacts when pressed.

24. The aerosol delivery device of claim 19, wherein the actuator assembly comprises:
a slider body slidably disposed within the main body of the holder;
an actuator coupled to the slider body and configured to move the slider body along a length of the main body in a first direction and a second direction;
a first contact arm pivotably coupled to the main body and configured to receive one of the pair of actuatable ignitor contacts, the first contact arm actuated via contact with the slider body when the slider body is moved in the first direction;
a second contact arm pivotably coupled to the main body and configured to receive the other one of the pair of actuatable ignitor contacts, the second contact arm actuated via contact with the slider body when the slider body is moved in the first direction; and
a biasing mechanism configured to engage the slider body and bias the slider body in the second direction,
wherein actuation of the first and second contact arms moves the ignitor contacts from the first position to the second position.

25. The aerosol delivery device of claim 17 further comprising an actuator assembly coupled to the holder and configured to move the cartridge between a loading position, a lighting position, and an ejecting position, wherein the actuator assembly comprises:
a slider body slidably disposed within the main body of the holder and defining a receptacle configured to at least partially receive the removable cartridge, the slider body configured to slide along a length of the main body in a first direction and a second direction;
an actuator coupled to the slider body and configured to move the actuator assembly between the loading position, the lighting position, and the ejecting position; and
a spring assembly configured to engage the slider body and bias the actuator assembly into the lighting position.

26. The aerosol delivery device of claim 17 further comprising an actuator assembly comprising:
a first button assembly movably disposed within the holder proximate a first one of the pair of actuatable ignitor contacts;
a first contact arm pivotably coupled to the main body and configured to receive the elongate body of the first ignitor contact, the first contact arm actuated via contact with the first button assembly so as to pivot the first ignitor contact from its first position to its second position;
a second button assembly movably disposed within the holder proximate the second one of the pair of actuatable ignitor contacts, the second button assembly oriented substantially in opposition to the first button assembly; and
a second contact arm pivotably coupled to the main body and configured to receive the elongate body of the second ignitor contact, the second contact arm actuated via contact with the second button assembly so as to pivot the second ignitor contact from its first position to its second position.

27. The aerosol delivery device of claim 26, wherein actuation of the button assemblies delivers electrical energy to the ignitable heat source.

28. The aerosol delivery device of claim 19, wherein the actuator assembly comprises:
a first button assembly movably disposed within the holder proximate a first one of the pair of actuatable ignitor contacts; and
a second button assembly movably disposed within the holder proximate the second one of the pair of actuatable ignitor contacts, the second button assembly oriented substantially in opposition to the first button assembly,
wherein actuation of the button assemblies moves the first and second actuatable ignitor contacts from their first positions to their second positions.

29. The aerosol delivery device of claim 28 further comprising:
a first contact arm pivotably coupled to the main body and configured to pivotably couple the first ignitor contact to the main body;
a second contact arm pivotably coupled to the main body and configured to pivotably couple the second ignitor contact to the main body;
wherein the first and second button assemblies are in contact with the first and second contact arms, respectively, such that actuation of the button assemblies pivots the first and second ignitor contacts into their second positions.

30. An aerosol delivery device comprising:
a holder comprising an upper body portion and a lower body portion, each defining a proximal end and a distal end, wherein:
the lower body portion further defines a receiving chamber disposed in the proximal end thereof and configured to receive a removable cartridge comprising an ignitable heat source and a first aerosol passageway, and
the upper body portion comprises a power source disposed therein and defines a second aerosol passageway therethrough, wherein the upper body portion and the lower body portion are movably coupled together;
a mouthpiece configured to engage with a user's mouth; and
a pair of actuatable ignitor contacts movably coupled to the lower body portion and disposed proximate the distal end thereof, wherein the actuatable ignitor contacts are configured to be engageable with the ignitable heat source when the removable cartridge is secured within the receiving chamber and movable between a first position spaced apart from the ignitable heat source and a second position contacting the ignitable heat source.

31. The aerosol delivery device of claim 30 further comprising an actuator assembly coupled to the lower body portion and configured to move the actuatable ignitor contacts between the first position and the second position.

32. The aerosol delivery device of claim 30, wherein the actuatable ignitor contacts are in electrical communication with the power source.

33. The aerosol delivery device of claim 30, wherein the actuator assembly comprises:
a first button assembly movably disposed within the lower body portion proximate a first one of the pair of actuatable ignitor contacts; and
a second button assembly movably disposed within the lower body portion proximate the second one of the pair of actuatable ignitor contacts, the second button assembly oriented substantially in opposition to the first button assembly,
wherein actuation of the button assemblies moves the first and second actuatable ignitor contacts from their first positions to their second positions.

34. The aerosol delivery device of claim 33, wherein each of first and second button assemblies is coupled to the holder via a biasing mechanism configured to maintain the first and second button assemblies in a neutral position.

35. The aerosol delivery device of claim 30 further comprising an indicator configured to indicate a state of the device

36. An aerosol delivery device comprising:
a holder comprising a main body defining a proximal end and a distal end, the main body further defining a receiving chamber configured to receive a removable cartridge comprising an ignitable heat source and a first passageway extending at least partially therethrough;
a removable mouthpiece assembly configured to engage the proximal end of the holder, the mouthpiece assembly defined by a first end and a longitudinally opposed second end and defining a second passageway extending therethrough, wherein the first end is configured to engage with a user's mouth and the second end is configured to sealingly engage and removably secure the removable cartridge therein, wherein the second end is disposable within the first passageway of the main body so as to position at least a portion of the cartridge within the receiving chamber;
a power source disposed within the main body; and
a pair of actuatable ignitor contacts disposed proximate the distal end of the main body and configured to be engaged with the ignitable heat source when the removable cartridge is secured within the receiving chamber, wherein the ignitor contacts are movably coupled to the holder and movable between a first position spaced apart from the ignitable heat source and a second position contacting the ignitable heat source.

37. The aerosol delivery device of claim 36 further comprising a first biasing mechanism engaged with the ignitor contacts and configured to bias the ignitor contacts into the first position.

38. The aerosol delivery device of claim 37, wherein each of the actuatable ignitor contacts comprises an elongate body and a contact portion disposed at a distal end thereof, the elongate body pivotably coupled to an end cap disposed within the distal end of the holder and wherein the contact portions are pivotable towards each other and into the second position.

39. The aerosol delivery device of claim 37 further comprising an actuator assembly disposed within the holder and configured to actuate the ignitor contacts between the first position and the second position.

40. The aerosol delivery device of claim 39, wherein the actuator assembly comprises:
a solenoid in electrical communication with the power source; and
a collar engageable with the solenoid and a proximal end of each of the elongate bodies of the ignitor contacts, wherein the collar is configured to be advanced into contact with the elongate bodies when the solenoid is energized so as to pivot the ignitor contacts into the second position.

41. The aerosol delivery device of claim 40, wherein the collar is configured to be retracted from the elongate bodies when the solenoid is de-energized so that the ignitor contacts return to the first position.

42. The aerosol delivery device of claim 40 further comprising:
a printed circuit board disposed within the holder and in electrical communication with the ignitor contacts; and
a switch in electrical communication with the power source and the printed circuit board and configured to electrically couple the power source to the ignitor contacts.

43. The aerosol delivery device of claim 42, wherein the switch is also configured to electrically couple the power source to the solenoid.

44. The aerosol delivery device of claim 36, wherein the removable mouthpiece assembly comprises:
a first portion comprising an elongate body defined by a first end and a longitudinally opposed second end and defining at least a portion of the second passageway;
a second portion defined by a first end and a longitudinally opposed second end and defining a first aerosol passageway therethrough, wherein the first end of the second portion is configured to engage with a user's mouth and the second end is partially disposed within the second passageway in the first portion of the mouthpiece assembly proximate the first end of the first portion;
a slider body defined by a first end and a longitudinally opposed second end and defining a second aerosol passageway therethrough, wherein the slider body is slidably disposed within the first portion of the mouthpiece assembly and the first end of the slider body is coupled to the second end of the second portion of the mouthpiece assembly and the second end of the slider body is configured to engage the removable cartridge;
an inner tubular body defined by a first end and a longitudinally opposed second end and defining a receptacle comprising a second biasing mechanism disposed therein, the receptacle configured to at least partially receive the slider body, the slider body slidably coupled to the inner tubular body, wherein the second end of the inner tubular body is configured to engage the first portion of the mouthpiece proximate the second end of the first portion and the second biasing mechanism is configured to maintain the slider body in a loading configuration; and
an outlet guide coupled to the second end of the first portion of the mouthpiece and partially received within the second end of the inner tubular body, wherein the outlet guide defines a cavity configured to sealingly engage and removably secure the removable cartridge therein.

45. The aerosol delivery device of claim 44, wherein the holder further comprises:
an inner housing disposed within the main body of the holder and defining one or more receptacles therein, a proximal end of the inner housing coupled to the proximal end of the holder and defining a first opening therethrough;
an outer tubular body disposed within a first one of the receptacles and defining at least a portion of the first passageway and configured to slidably receive the mouthpiece assembly therein;
an end cap coupled to the distal end of the holder and defining a second opening in communication with the first passageway of the outer tubular body, the end cap configured to secure at least one of the inner housing or the outer tubular body within the holder.

46. The aerosol delivery device of claim 45, wherein the holder comprises a retention mechanism configured to movably couple the mouthpiece assembly to the holder.

47. The aerosol delivery device of claim 45, wherein the end cap further comprises a standoff extending into the inner housing, the stand-off partially defining the receiving chamber and configured to pivotably engage the actuatable ignitor contacts.

48. The aerosol delivery device of claim 42, wherein the printed circuit board further comprises a charging port and the printed circuit board is oriented within the inner housing so that the charging port is disposed through a side wall of the holder.

49. The aerosol delivery device of claim 44, wherein the second end of the slider body comprises a stem configured to engage with and eject the removable cartridge upon application of a force to the second portion of the mouthpiece.
